(19) United States
(12) Patent Application Publication
(10) Pub. No.: US 2026/0191973 A1
(43) Pub. Date: Jul. 9, 2026

(54) STIMULI-RESPONSIVE ENGINEERING PLATFORM FOR INTRACELLULAR PAYLOAD DELIVERY

(71) Applicants: Couragene, Inc., North Brunswick, NJ (US); Yale University, New Haven, CT (US)

(72) Inventors: Jiangbing Zhou, Cheshire, CT (US); Zefeng Wang, New Haven, CT (US); Jiang Yu, New Haven, CT (US); Jiali Fan, New Haven, CT (US); Zewei Tu, New Haven, CT (US); Conrad Leung, North Brunswick, NJ (US); Amy Liao, North Brunswick, NJ (US); Min-Han Lin, North Brunswick, NJ (US); Ying Xiao, North Brunswick, NJ (US); Ying Xie, North Brunswick, NJ (US); Huacan Lin, Tianjin (CN)

(21) Appl. No.: 19/443,130

(22) Filed: Jan. 8, 2026

Related U.S. Application Data
(60) Provisional application No. 63/743,367, filed on Jan. 9, 2025.

(51) Int. Cl. A61K 47/60; A61K 48/00; C08G 65/334

(52) U.S. Cl. CPC A61K 47/60; A61K 48/0008; C08G 65/3348

(57) ABSTRACT

Compounds having the following structure:

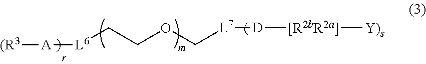

wherein Y=

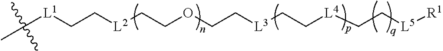

wherein: [R^{2b}R^{2a}] represents a reaction product between R^{2b} and R^{2a} of Formulas (2) and (1), respectively, R³ functions as a cell penetration moiety, and R¹ functions as a binding moiety to bind to the payload (P) to form a conjugate with P, wherein R¹, R³, A, D, L¹, L², L³, L⁴, L⁵, L⁶, L⁷, r, m, s, n, p, and q are as defined in further detail elsewhere in this disclosure. Also described herein are methods of producing compounds of Formulas (1), (2), and (3), conjugates thereof with a payload (P), pharmaceutical compositions thereof, methods of delivering the conjugates to subjects or cells, and methods of treating a disease or condition by administering such conjugates.

### CROSS REFERENCE TO RELATED APPLICATIONS

This application claims the benefit of priority from U.S. Provisional Application No. 63/743,367, filed on Jan. 9, 2025, which is herein incorporated by reference in its entirety.

### STATEMENT REGARDING FEDERALLY SPONSORED RESEARCH

This invention was made with government support under Contract No. NS115597 awarded by the National Institutes of Health (NIH). The government has certain rights in the invention.

### FIELD OF THE INVENTION

The present invention generally relates to payload delivery, particularly the intracellular delivery of payloads using payload-membrane penetrating conjugates in which the payload and membrane-penetrating portion of the conjugate are covalently or non-covalently linked, in some cases via a stimuli-responsive linker.

### BACKGROUND

Clinical translation of biologics-based therapies, such as protein-based and gene-based therapies, has been a major challenge, because most biologics (e.g., gene-based and/or protein therapeutics) cannot efficiently penetrate the cell. Although significant efforts have focused on developing biologics for clinical use, significant obstacles exist in the development of effective systems for payload delivery. These include penetrating the cell membrane, release of payload, effective such release such that the payload retains its biological activity, and/or release of payload in the absence of carrier molecules covalently attached to the payload. Accordingly, there remains a need for improved compositions and methods for the effective intracellular delivery of biologics.

### SUMMARY

The present disclosure is foremost directed to conjugates that confer improved intracellular deliver of biologics. It is a further object of the invention to provide conjugates capable of passing through the cell membrane while retaining the biological activity of the biologics being delivered. It is another object of the invention to provide conjugates capable of delivering gene editing machinery into a cell. It is yet a further object of the invention to provide methods for using such conjugates.

Described herein are bifunctional molecules, conjugates thereof with payloads, and pharmaceutical compositions containing these conjugates for intracellular delivery of a payload. The conjugates contain the payload, a chemical linker, and a cell membrane penetrating moiety. Some preferred payloads for delivery are gene editing machineries. The cell membrane penetrating moiety facilitates intracellular uptake of the conjugates. In some embodiments, the chemical linker contains a stimuli-responsive chemical moiety and/or a self-immolative chemical moiety. Upon entry into a cell, the conjugate is exposed to one or more stimuli, such as a reducing and/or an acidic environment, that cleave the stimuli-responsive chemical moiety, thereby activating self-immolation of the chemical linker. The cleavage and self-immolation lead to the delivery of the payload in a stimuli-responsive traceless manner.

The results described herein demonstrate that the platform can be used, in particular, for the effective intracellular delivery of ribonucleoproteins (RNPs) composed of Cas9 protein and sgRNA in in vitro cell cultures and in vivo in both reporter and diseased models. The results also show that chemically modified RNPs, termed stimuli-responsive traceless engineering platform RNPs (STEP RNPs), are safe as intravenous administration of STEP RNPs and do not induce significant systemic toxicity to the liver and kidney based on aspartate transaminase (AST), alanine transaminase (ALT), blood urea nitrogen (BUN), and creatine assays. Further, intracranial administration of STEP RNPs does not induce significant cellular damage to the brain based on microscopic imaging and by H&E staining. Storage of the STEP RNPs at −20° C. over 2 months does not reduce their efficiency in genome editing.

In a first aspect, the present disclosure is directed to bifunctional molecules containing a binding domain (R¹) and having the following structure:

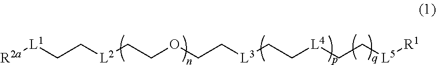

wherein: R^{2a} is a first reactive group that forms a linkage with a second reactive group; L¹ is a linker selected from a bond, C(O), CH₂, C(O)NH, and C(O)O; L², L⁴, and L⁵ are linkers independently selected from a bond, CH₂, C(O), NH, O, S, C(O)NH, C(O)O, OC(O)NH, OC(O)O, NHC(O)NH, and —OC(O)O—Ar—, or a CH₂ bound to any of the foregoing linkers, wherein Ar is an aromatic ring, and wherein an O atom can optionally be replaced with an S atom in any of the foregoing linkers; L³ is a linker selected from the group consisting of a bond, CH₂, CH₂CH₂, S—S, C(O)O, OC(O)O, C(O)NH, OC(O)O—Ar, wherein Ar is an aromatic ring, and linked combinations of any two or more of the foregoing linkers; R¹ (which functions as a binding domain) is an aromatic or aliphatic ring or polycyclic ring system, wherein the aromatic or aliphatic ring or polycyclic ring system optionally contains one or more ring heteroatoms selected from N, O, and S, and the aromatic or aliphatic ring or polycyclic ring system optionally contains one or more non-H substituents selected from halogen atoms; oxo; linear or branched, cyclic or acyclic, saturated or unsaturated, halogenated or non-halogenated hydrocarbon groups containing 1-30 carbon atoms; NO₂; NR³R⁴; CN; C(O)R⁵; C(O)OR⁶; OC(O)R⁶; OR⁷; C(O)NR³R⁴; and NHC(O)R⁸; wherein R³, R⁴, R⁵, R⁶, R⁷, and R⁸ are independently selected from H and linear or branched alkyl groups containing 1-3 carbon atoms; and R¹ is optionally connected to L⁵ by a CR^{a}R^{b} linker, wherein R^{a} and R^{b} are independently selected from H; R⁹; OR⁹; NR¹⁰R¹¹; CN; C(O)R¹²; C(O)OR¹³; OC(O)R¹⁴, C(O)NR¹⁰R¹¹; and NHC(O)R¹⁵; wherein R⁹, R¹⁰, R¹¹, R¹², R¹³, R¹⁴, and R¹⁵ are independently selected from H and alkyl, alkenyl, and cycloalkyl groups containing 1-6 carbon atoms and optionally containing one or halogen atoms; n is selected from an integer in the range of 0-100; p is an integer of 0-10; and q is an integer of 0-50.

In a second aspect, the present disclosure is directed to bifunctional molecules containing a cell penetration moiety (R³) and having the following structure:

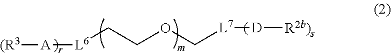

wherein: R^{2b} is a second reactive group that forms a linkage with a first reactive group; r and s are independently selected from an integer in the range of 1-6; L⁶ is either: (i) a non-branching linker selected from CH₂, C(O), NH, O, S, C(O)NH, C(O)O, OC(O)NH, OC(O)O, and NHC(O)NH, or one or more of these attached together, wherein r is 1; or (ii) a branching linker selected from C, CH, N, B, or a linear or branched, cyclic or acyclic hydrocarbon linker group R^{c} containing 2-50 carbon atoms on which an r number of R³-A arms are attached, wherein R^{c} optionally contains one or more heteroatoms selected from N, O, and S; or (iii) one or more non-branching linkers in (i) attached to a branching linker (ii); L⁷ is either: (i) a non-branching linker selected from CH₂, C(O), NH, O, S, C(O)NH, C(O)O, OC(O)NH, OC(O)O, and NHC(O)NH, in which case s is 1; or (ii) a branching linker selected from C, CH, N, B, or a linear or branched, cyclic or acyclic hydrocarbon linker group Re containing 2-50 carbon atoms on which an s number of R^{2b}-D arms are attached, wherein R^{c} optionally contains one or more heteroatoms selected from N, O, and S, or (iii) one or more non-branching linkers in (i) attached to a branching linker (ii); A is a bond or a linker containing 1-50 carbon atoms and optionally containing one or more heteroatoms selected from N, O, and S; D is a bond or a linker containing 1-50 carbon atoms and optionally containing one or more heteroatoms selected from N, O, and S; R³ (the cell penetration moiety) is independently a linear or branched, cyclic or acyclic, saturated or unsaturated hydrocarbon group containing 4-30 carbon atoms optionally containing one or more heteroatoms selected from N, O, S, and B and which functions as a cell penetration moiety; and m is selected from an integer in the range of 0-100.

In a third aspect, the present disclosure is directed to monofunctional molecules derived from molecules of Formulas (1) and (2) wherein first and second reactive groups (R^{2a} and R^{2b}, respectively) have reacted to form a linkage reaction product denoted as [R^{2b}R^{2a}]. The monofunctional molecules have the following structure:

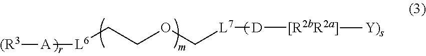

wherein Y=

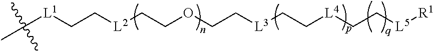

wherein: R¹, R³, A, D, L¹, L², L³, L⁴, L⁵, L⁶, L⁷, r, m, s, n, p, and q are as defined above, and [R^{2b}R^{2a}] represents a reaction product between R^{2b} and R^{2a}.

In a fourth aspect, the present disclosure is directed to conjugates of a payload (P) with molecules of Formula (3), wherein the conjugates have the following structure:

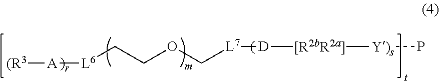

wherein Y′ is a linkage having the following structure:

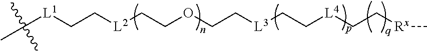

wherein: R¹, R³, A, D, L¹, L², L³, L⁴, L⁵, L⁶, L⁷, r, m, s, n, p, and q are as defined above, and [R^{2b}R^{2a}] represents a reaction product between R^{2b} and R^{2a}; P is a peptide-containing or nucleic acid-containing molecule; R^{x} is a linkage reaction product between L⁵-R¹, as defined in claim 1, and a group or domain on the P molecule; the dashed bond indicates a covalent or non-covalent bond; and t is at least 1. In some embodiments, P is a peptide or a protein, and the peptide or protein contains at least 50 amino acids, at least 75 amino acids, at least 100 amino acids, or less than 50 amino acids, or a molecular weight of 10-500 kDa, 30-300 kDa, 50-300 kDa, 30-250 kDa, or 50-250 kDa.

In a fifth aspect, the present disclosure is directed to conjugates of a payload (P) with a molecule of Formula (1) containing a binding domain (R¹), wherein the conjugates have the following structure:

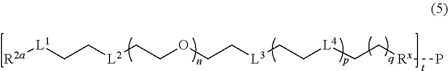

wherein: R^{2a}, L¹, L², L³, L⁴, n, p, and q are as defined earlier above; P is a peptide-containing or nucleic acid-containing molecule; R^{x} is a linkage reaction product between L⁵-R¹, as defined above under Formula (1), and a group on the P molecule; the dashed bond indicates a covalent or non-covalent bond; and t is at least 1 (or at least 2, 3, 4, or 5).

In a sixth aspect, the present disclosure is directed to a method of producing molecules of Formula (3), the method comprising:

reacting a molecule of the following formula:

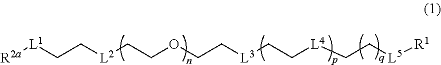

with a molecule of the following formula:

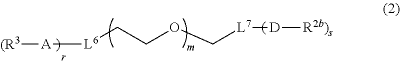

under conditions in which R^{2a} forms a linkage with R^{2b} to result in the following molecule:

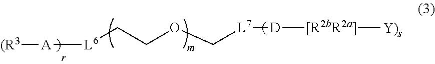

wherein Y=

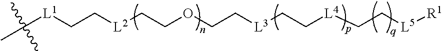

wherein R¹, R³, A, D, L¹, L², L³, L⁴, L⁵, L⁶, L⁷, r, m, s, n, p, and q are as defined earlier above, and [R^{2b}R^{2a}] represents a reaction product between R^{2b} and R^{2a}

In a seventh aspect, the present disclosure is directed to a one-step or two-step method of producing conjugates of a payload (P) with molecules of Formula (3), the method comprising:

for the one-step approach:
reacting a payload molecule (P) with a molecule of the following formula:

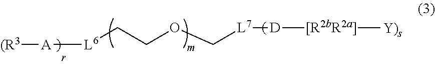

wherein Y=

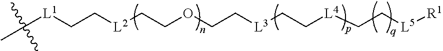

under conditions in which R¹ reacts with a group in P to result in the following molecule:

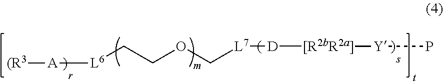

wherein Y′ is a linkage having the following structure:

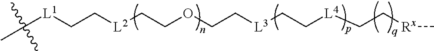

wherein: R¹, R³, A, D, L¹, L², L³, L⁴, L⁵, L⁶, L′, r, m, s, n, p, and q are as defined earlier above, and [R^{2b}R^{2a}] represents a reaction product between R^{2b} and R^{2a}; P is a peptide-containing or nucleic acid-containing molecule; R^{x} is a linkage reaction product between L⁵-R¹, as defined in claim 1, and a group on the P molecule; the dashed bond indicates a covalent or non-covalent bond; and t is at least 1;
or for the two-step approach:
in a first step, reacting a payload molecule (P) with a molecule of the following formula:

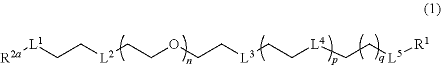

under conditions in which R¹ reacts with a group or domain in P to result in the following molecule:

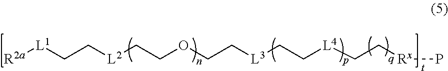

wherein: R^{2a}, L¹, L², L³, L⁴, n, p, and q are as defined earlier above; P is a peptide-containing or nucleic acid-containing molecule; R^{x} is a linkage reaction product between L⁵-R¹, as defined under Formula (1), and a group on the P molecule; the dashed bond indicates a covalent or non-covalent bond; t is at least 1; and in a second step, reacting the conjugate molecule of Formula (5) with a molecule of the Formula (2) having the following structure:

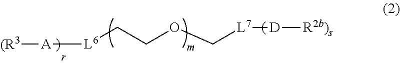

under conditions in which R^{2a} (a second reactive group) forms a linkage with R^{2b} to result in the following molecule of Formula (4):

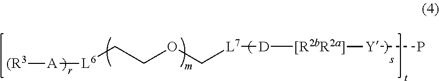

wherein Y′ is a linkage having the following structure:

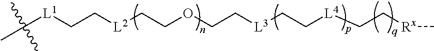

wherein: R¹, R³, A, D, L¹, L², L³, L⁴, L⁵, L⁶, L⁷, r, m, s, n, p, and q are as defined earlier above, and [R^{2b}R^{2a}] represents a reaction product between R^{2b} and R^{2a}, P is a peptide-containing or nucleic acid-containing molecule; R^{x} is a linkage reaction product between L⁵-R¹, as defined under Formula (1), and a group on the P molecule; the dashed bond indicates a covalent or non-covalent bond; and t is at least 1.

In an eighth aspect, the present disclosure is directed to a method of producing a conjugate of a molecule of Formula (1) with a payload (P), the method comprising:

reacting a payload molecule (P) with a molecule of the following formula:

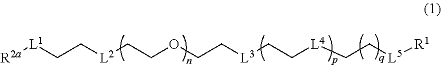

under conditions in which R¹ reacts with a group in P to result in the following molecule:

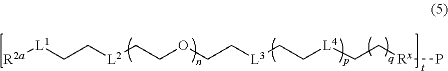

wherein: R^{2a}, L¹, L², L³, L⁴, n, p, and q are as defined under Formula (1); P is a peptide-containing or nucleic acid-containing molecule; R^{x} is a linkage reaction product between L⁵-R¹, as defined under Formula (1), and a group on the P molecule; the dashed bond indicates a covalent or non-covalent bond; and t is at least 1.

In a ninth aspect, the present disclosure is directed to a method of delivering a payload (P) to a cell by contacting the conjugate of Formula (4) containing a cell penetration moiety (R³) to a cell, the method comprising:

contacting or interacting a conjugate molecule of the Formula (4) with a cell with resulting penetration of the conjugate molecule of the Formula (4) into the cell by means of the cell penetration moiety R³, wherein the conjugate molecule of Formula (4) has the following formula:

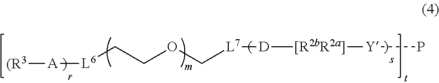

wherein Y′ is a linkage having the following structure:

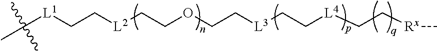

wherein: R¹, R³, A, D, L¹, L², L³, L⁴, L⁵, L⁶, L⁷, r, m, s, n, p, and q are as defined earlier above, and [R^{2b}R^{2a}] represents a reaction product between R^{2b} and R^{2a}; P is a peptide-containing or nucleic acid-containing molecule; R^{x} is a linkage reaction product between L⁵-R¹, as defined under Formula (1), and a group on the P molecule; the dashed bond indicates a covalent or non-covalent bond; and t is at least 1.

In a tenth aspect, the present disclosure is directed to pharmaceutical compositions comprising any of the conjugates described above and a pharmaceutically acceptable carrier. In one embodiment, the conjugate has the composition of Formula (4). In another embodiment, the conjugate has the composition of Formula (5).

In an eleventh aspect, the present disclosure is directed to a method of delivering a peptide-containing or nucleic acid-containing payload molecule (P) to a subject in need thereof, the method comprising administering any of the above conjugates or a pharmaceutical composition described above to the subject. In one embodiment, the conjugate has the composition of Formula (4). In another embodiment, the conjugate has the composition of Formula (5).

In a twelfth aspect, the present disclosure is directed to a method of treating a disease or condition in a subject, the method comprising: administering any of the conjugates described above or a pharmaceutical composition described above to the subject, wherein the payload (P) in the conjugate is a peptide-containing or nucleic acid-containing molecule. In one embodiment, the conjugate has the composition of Formula (4). In another embodiment, the conjugate has the composition of Formula (5).

### BRIEF DESCRIPTION OF THE FIGURES

FIG. 1 is a scheme showing a proposed mechanism of the stimuli-responsive traceless engineering platform (STEP) for intracellular payloads delivery, as described in the present disclosure.

FIG. 2 shows molecular structures of compounds of Formula (1) containing aromatic binding domains corresponding to R¹.

FIGS. 3A-3B. FIG. 3A is a schematic representation of Ai9 loxP-flanked STOP reporter cassette. FIG. 3B is a graph comparing the gene editing efficiency of Cas9/sgAB RNPs (ribonucleoproteins) delivered by STEP with the different aromatic binding domains shown in FIG. 2. Ai9 fibroblast cells were treated with indicated RNPs/STEP at Cas9 concentration of 10 μg/mL. 48 h after treatment, the cells were analyzed under fluorescence microscope, based on which the percentage of cells expressing tdTomato was quantified.

FIG. 4 shows molecular structures of compounds of Formula (1) containing non-aromatic binding domains corresponding to R¹.

FIG. 5 is a graph comparing gene editing efficiency of Cas9/sgAB RNPs delivered by STEP with the different nonaromatic binding domains shown in FIG. 4. Ai9 fibroblast cells were treated with indicated RNPs/STEP at Cas9 concentration of 10 μg/mL. 48 h after treatment, the cells were analyzed under fluorescence microscope, based on which the percentage of cells expressing tdTomato was quantified.

FIGS. 6A-6D. The binding activity of R¹ molecules to Cas9 RNP. FIG. 6A is a schematic showing no covalent conjugation of STEP NPC to Cas9. CLS-NPC STEP (molar ratio 20 to Cas9) did not chemically react with Cas9 (1 mg/mL) to form the byproduct with absorbance at 405 nm in pH7.4 buffer. CLS-NPC STEP did not react with negative control PBS. CLS-NPC STEP interacted with positive control NaOH (0.1 M) to produce absorbance at 405 nm. FIG. 6B shows chemical structures of various STEPs terminated with a nitrophenyl carbonate (NPC) group, 4-nitrophenyl acetate (NPA) or —OH group. FIG. 6C is a graph showing results of biolayer interferometry (BLI) analysis of the binding activity between STEP to Cas9. BLI-derived steady-state analysis of the binding response (nm) as a function of chemicals concentration (μM). FIG. 6D shows gene editing activity of various STEP RNP in Ai9 reporter cells (Cas9 10 μg/mL).

FIG. 7 shows molecular structures of various aromatic membrane penetration molecules corresponding to R³.

FIG. 8 is a graph comparing various aromatic R³ molecules on gene editing efficiency in Ai9 reporter cells for compounds shown in FIG. 7

FIG. 9 shows molecular structures of various non-aromatic membrane penetration molecules corresponding to R³.

FIG. 10 is a graph comparing various non-aromatic R³ molecules for gene editing efficiency in Ai9 reporter cells for compounds shown in FIG. 9.

FIGS. 11A-11B. Effect of traceless linkers on gene editing activity. FIG. 11A shows chemical structures of traceless linkers (—S—S—) and paired untraceless linkers (—C—C—) with 3 different binding molecules including -NPC, -X, and -Y. FIG. 11B shows their respective gene editing delivery efficiencies in Ai9 reporter cells (Cas9 10 μg/mL). Among the three pairs of linkers, all of the traceless linkers (—S—S—) showed significantly higher editing efficiency than their paired untraceless linkers (—C—C—).

FIG. 12 shows a number of exemplary branched molecular structures (with s>1 or r>1) as follows: BM-1, BM-2, BM-3, and BM-4 are molecules corresponding to Formula (3) in which s=2, 2, 3, and 3, respectively (and r is 1 for all, and all contain R³ as cholesterol). BM-5, BM-6, BM-7, and BM-8 are molecules corresponding to Formula (2) in which s=3 and R^{2b} is azide (N₃). BM-9, BM-10, BM-11, and BM-12 are molecules corresponding to Formula (3) in which r=3 and s=1 (with all containing R³ as linear unsaturated or saturated cell penetration moieties).

FIG. 13 is a graph comparing gene editing efficiency in Ai9 reporter cells for compounds shown in FIG. 12.

FIG. 14 shows the effect of the linker length on the gene editing activity. Editing efficacy: +, 0-10%; ++, 10-30%; +++, 30-100%.

FIG. 15 shows a number of exemplary molecular structures showing possible R^{2a} and R^{2b} reactive groups to link R¹ (binding domain) and R³ (cell penetration) groups in a single molecule capable of binding to a payload (P) through R¹.

FIG. 16 is a graph comparing the gene editing activity for compounds shown in FIG. 15.

FIG. 17 is a graph comparing different formulation conjugation methods (i.e., one-step or two-step, as further described in detail elsewhere in the present disclosure) on gene editing efficiency in Ai9 reporter cells.

FIG. 18 is a graph showing the delivery of Cre by STEP in Ai9 reporter cells. The data show that STEP-Cre can effectively edit over 98% Ai9 reporter cells at 2 μg/mL, while free Cre did not edit Ai9 cells at 2 μg/mL.

FIG. 19 is a graph showing STEP is capable of delivering antibodies into cells. IgG antibody was conjugated to STEP and added to NF-κB-RE-luc2P HEK293 cells. TNF-α were added to induce reporter gene expression. After 5 hours of induction in a tissue culture incubator, luciferase activity was quantified using the Luciferase Assay System Reagent. The results showed that STEP-1, which is composed of β-sitosterol and NPC, was able to effectively deliver the antibody into cells to target cellular NF-κB signaling.

FIGS. 20A-20F. FIG. 20A is a schematic diagram showing dCas9-Tet1CD (upper panel) and dCas9-JMJD2a (lower panel) targeting methylated CpG and H3K9me2/3, respectively. FIG. 20B is a schematic showing the loci of sgRNA binding to Prader-Willi Syndrome (PWS)-imprinting center or around the region including CpG islands. Maternal imprinted/silenced gene also shown. FIGS. 20C-20F are bar graphs of RT-qPCR analysis showing the reactivation of maternal imprinted SNRPN gene in human fibroblasts derived from PWS (paternal deletion of 15q11-q13) by RNP including dCas9-Tet1CD with sgRNA (FIG. 20C) and RT-qPCR analysis showing the reactivation of imprinted SNRPN, SNORD116, and 116HG genes by dCas9-JMJD2a with sgRNA #4 in human fibroblasts derived from PWS (FIGS. 20D-20F).

FIGS. 21A-1B. The internalization and intracellular trafficking of STEP RNP in cells. FIG. 21A is a graph showing the effect of endocytosis inhibitors and temperature on the internalization of STEP GFP-Cas9/sgNC RNP in cells. Cells were pretreated with various endocytosis inhibitors PitStop (50 μM), Nystatin (100 μM), Amiloride (0.5 mM) or 4° C. incubation for 30 min and then treated with STEP GFP-Cas9 RNP for 4 hours. Quantification of cell internalization is shown by mean fluorescence intensity (MFI) through flow cytometric analyses of cells. FIG. 21B shows confocal microscopy images of intracellular trafficking of STEP GFP-Cas9/sgNC RNP in cells after incubation with cells for different times. Cells were stained by EEA1 antibody and DAPI.

FIG. 22 shows fluorescent images of tdTomato expression in Ai9 reporter cells after treatment with STEP RNP where binding domain R¹ is NPC, cell penetrating domain R³ is cholesterol, carrying sgRNA targeting Ai9 (sgAB) or scramble sgRNA (sgNC) at Cas9 concentration of 15 μg/mL

FIG. 23 is a graph plotting the hydrodynamic size and zeta-potential of RNP and STEP-RNP by dynamic light scattering measurement.

FIGS. 24A-24B. The size and morphology of STEP RNP. FIG. 24A is a graph plotting the size of STEP RNP in PBS as determined by dynamic light scattering analysis. The dynamic size is around 13 nm with a polydispersity index of 0.2. FIG. 24B shows transmission electron microscopy (TEM) images of RNP and STEP-RNP. No apparent size and morphology differences were observed between RNP and STEP RNP.

As FIG. 25 illustrates the working mechanism of the GFP-on (GER10) mouse model, which is used to assess the efficiency of both base and prime editing.

FIGS. 26A-26D. The editing effect of STEP ABE-RNP in GER10 NPC cells. FIG. 26A depicts the denaturing polyacrylamide gel electrophoresis (PAGE) band of the purified ABE8e-NG protein. FIG. 26B shows HL1172 ABE-RNP-edited GER10 NPC cells exhibiting green fluorescence, compared to a negative control (ABE-RNP) that produced no fluorescent signal. FIG. 26C shows that GER10 NPC cells were edited by HL1172 ABE-RNP, achieving an editing efficiency of approximately 15.43% as measured by flow cytometry in one experiment. FIG. 26D shows the average editing efficiency of HL1172 ABE-RNP in GER10 NPC cells, approximately 13%, based on a quadruplicate experiment.

FIGS. 27A-27B. The prime editing effect of STEP PE-RNP in NPC cells. FIG. 27A depicts the denaturing polyacrylamide gel electrophoresis (PAGE) band of the semi-purified prime editor 7 (PE7) protein. FIG. 27B shows GER10 NPC cells edited with STEP-PE-RNP exhibiting green fluorescence, compared to a negative control.

### DETAILED DESCRIPTION

### Hydrocarbon Groups

As used herein, the term “hydrocarbon group” (also denoted by the group R) is defined as a chemical group containing at least carbon and hydrogen atoms. In some embodiments, R is composed solely of carbon and hydrogen. In other embodiments, the hydrocarbon group contains carbon, hydrogen, and optionally, one or more fluorine atoms to result in partial or complete fluorination of the hydrocarbon group. In different embodiments, one or more of the hydrocarbon groups or linkers can contain precisely, or a minimum of, or a maximum of, for example, 1, 2, 3, 4, 5, 6, 7, 8, 9, 10, 12, 15, 18, 20, 22, 24, 26, 28, 30, 40, or 50 carbon atoms, or a number of carbon atoms within a particular range bounded by any two of the foregoing carbon numbers (e.g., 1-50, 1-40, 1-30, 1-20, 1-12, 1-8, 1-6, 1-5, 1-4, 1-3, 2-50, 2-40, 2-30, 2-20, 2-12, 2-8, 2-6, 2-5, 2-4, or 2-3 carbon atoms). Hydrocarbon groups in different compounds described herein, or in different generic groups of a compound, may possess the same or different number (or preferred range thereof) of carbon atoms. For example, as further discussed below, any one of R¹, R², R³, R⁴, R⁵, R⁶, R^{a}, R^{b}, R^{c}, and R^{d} in any of the generic formulas disclosed herein may independently contain a number of carbon atoms within any of the ranges provided above.

In a first set of embodiments, the hydrocarbon group (R) is a saturated and straight-chained group, i.e., a straight-chained (linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-eicosyl, n-docosyl, n-tetracosyl, n-hexacosyl, n-octacosyl, and n-triacontyl groups.

In a second set of embodiments, the hydrocarbon group (R) is saturated and branched, i.e., a branched alkyl group. Some examples of branched alkyl groups include isopropyl(2-propyl), isobutyl(2-methylprop-1-yl), sec-butyl(2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, and 1,2,2-trimethylprop-1-yl groups, isoheptyl, isooctyl, and the numerous other branched alkyl groups having up to 20 or 30 carbon atoms, wherein the “1-yl” suffix represents the point of attachment of the group.

In a third set of embodiments, the hydrocarbon group (R) is saturated and cyclic, i.e., a cycloalkyl group. Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

In a fourth set of embodiments, the hydrocarbon group (R) is unsaturated and straight-chained, i.e., a straight-chained (linear) olefinic or alkenyl group. The unsaturation occurs by the presence of one or more carbon-carbon double bonds and/or one or more carbon-carbon triple bonds. Some examples of straight-chained olefinic groups include vinyl, propen-1-yl (allyl), 3-buten-1-yl (CH₂—CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl(2-propynyl), 3-butynyl, and the numerous other straight-chained alkenyl or alkynyl groups having up to 20 or 30 carbon atoms.

In a fifth set of embodiments, the hydrocarbon group (R) is unsaturated and branched, i.e., a branched olefinic or alkenyl group. Some examples of branched olefinic groups include propen-2-yl (CH₂—C.—CH₃), 1-buten-2-yl (CH₂—C.—CH₂—CH₃), 1-buten-3-yl (CH₂═CH—CH.—CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, and the numerous other branched alkenyl groups having up to 20 or 30 carbon atoms, wherein the dot in any of the foregoing groups indicates a point of attachment.

In a sixth set of embodiments, the hydrocarbon group (R) is unsaturated and cyclic, i.e., a cycloalkenyl group. The unsaturated cyclic group can be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group may or may not also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems. All of the foregoing cyclic groups are carbocyclic groups.

One or more of the hydrocarbon groups (R) may also include one or more heteroatoms (i.e., non-carbon and non-hydrogen atoms), such as one or more heteroatoms selected from oxygen, nitrogen, sulfur, silicon, phosphorus, boron, and halogen atoms, as well as groups containing one or more of these heteroatoms (i.e., heteroatom-containing groups). The heteroatom-containing groups may be present as substituents in any of the hydrocarbon groups described herein. Some examples of oxygen-containing groups include hydroxy (OH), alkoxy (OR′), carbonyl-containing (e.g., carboxylic acid, ketone, aldehyde, carboxylic ester, amide, and urea functionalities), nitro (NO₂), carbon-oxygen-carbon (ether), sulfonyl, and sulfinyl (i.e., sulfoxide) groups, wherein R′ is H or a hydrocarbon group containing 1-12 (or 1-6, 1-4, or 1-3) carbon atoms. Some particular examples of alkoxy groups-OR′ include methoxy, ethoxy, n-propoxy, isopropoxy, n-butoxy, isobutoxy, t-butoxy, phenoxy, benzyloxy, 2-hydroxyethoxy, 2-methoxyethoxy, 2-ethoxyethoxy, vinyloxy, and allyloxy groups. In the case of an ether group, the ether group can also be a polyalkyleneoxide (polyalkyleneglycol) group, such as a polyethyleneoxide group. Some examples of nitrogen-containing groups include primary amine, secondary amine, tertiary amine (i.e., —NR² or NR³¹, wherein R is independently selected from H and hydrocarbon groups set forth above), nitrile, amide (i.e., —C(O)NR² or —NRC(O)R, wherein R is independently selected from hydrogen atom and hydrocarbon groups set forth above), imine (e.g., —CR═NR, wherein R is independently H or a hydrocarbon group), oxime (—CR═N—OH), amidoxime (—C(NH₂)═N—OH), nitro, urea (—NR—C(O)—NR², wherein R is independently H or a hydrocarbon group), and carbamate groups (—NR—C(O)—OR, wherein R is independently H or a hydrocarbon group). Some examples of phosphorus-containing groups include —PR₂, —PR₃⁺, —P(═O)R², —P(OR)₂, —O—P(OR)₂, —R—P(OR)₂, —P(═O)(OR)₂, —O—P(═O)(OR)₂, —O—P(═O)(OR)(R), —O—P(═O)R², —R—P(═O)(OR)₂, —R—P(═O)(OR)(R), and —R—P(═O)R² groups, wherein R is independently selected from hydrogen atom and hydrocarbon groups set forth above. Some examples of sulfur-containing groups include mercapto (i.e., —SH), thioether (i.e., sulfide, e.g., —SR), disulfide (—R—S—S—R), sulfoxide (—S(O)R), sulfone (—SO₂R), sulfonate (—S(═O)₂OR, wherein R is H, a hydrocarbon group, or a cationic group), and sulfate groups (—OS(═O)₂OR, wherein R is H, a hydrocarbon group, or a cationic group). Some examples of halide atoms include fluorine, chlorine, bromine, and iodine. In some embodiments, one or more of any of the heteroatoms described above (e.g., oxygen, nitrogen, and/or sulfur atoms) or heteroatom groups are inserted between carbon atoms (e.g., as —O—, —NR—, or —S—) in any of the hydrocarbon groups described above to form a heteroatom-substituted hydrocarbon group. Alternatively, or in addition, one or more of the heteroatom-containing groups can replace one or more hydrogen atoms in the hydrocarbon group.

In some embodiments, the hydrocarbon group (R) is or includes a cyclic or polycyclic group that includes at least one ring heteroatom (for example, one, two, three, four, or higher number of heteroatoms). Such ring heteroatom-substituted cyclic groups are referred to herein as “heterocyclic groups.” As used herein, a “ring heteroatom” is an atom other than carbon and hydrogen (typically, selected from nitrogen, oxygen, and sulfur) that is inserted into, or replaces a ring carbon atom in, a hydrocarbon ring structure. In some embodiments, the heterocyclic group is saturated, while in other embodiments, the heterocyclic group is unsaturated. An unsaturated heterocyclic group may be aliphatic or aromatic, wherein an aromatic heterocyclic group is also referred to herein as a “heteroaromatic ring,” or a “heteroaromatic fused-ring system” in the case of at least two fused rings, at least one of which contains at least one ring heteroatom. In some embodiments, the heterocyclic group is bound via one of its ring carbon atoms to another group (i.e., other than hydrogen atom and adjacent ring atoms), while the one or more ring heteroatoms are not bound to another group. In other embodiments, the heterocyclic group is bound via one of its heteroatoms to another group, while ring carbon atoms may or may not be bound to another group.

Some examples of saturated heterocyclic groups (R) containing at least one oxygen atom include oxetane, tetrahydrofuran, tetrahydropyran, 1,4-dioxane, 1,3-dioxane, and 1,3-dioxepane rings. Some examples of saturated heterocyclic groups (R) containing at least one nitrogen atom include pyrrolidine, piperidine, piperazine, imidazolidine, azepane, and decahydroquinoline rings. Some examples of saturated heterocyclic groups (R) containing at least one sulfur atom include tetrahydrothiophene, tetrahydrothiopyran, 1,4-dithiane, 1,3-dithiane, and 1,3-dithiolane rings. Some examples of saturated heterocyclic groups (R) containing at least one oxygen atom and at least one nitrogen atom include morpholine and oxazolidine rings. An example of a saturated heterocyclic group containing at least one oxygen atom and at least one sulfur atom includes 1,4-thioxane. An example of a saturated heterocyclic group containing at least one nitrogen atom and at least one sulfur atom includes thiazolidine and thiamorpholine rings. Saturated heterocyclic linkers (R) can be derived from any of the foregoing saturated heterocyclic groups by removal of one, two, or three hydrogen atoms from the saturated heterocyclic group to result in a divalent, trivalent, or tetravalent saturated heterocyclic linker, respectively.

Some examples of unsaturated heterocyclic groups (R) containing at least one oxygen atom include furan, pyran, 1,4-dioxin, benzofuran, dibenzofuran, and dibenzodioxin rings. Some examples of unsaturated heterocyclic groups (R) containing at least one nitrogen atom include pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, 1,4,5,6-tetrahydropyrimidine, 1,2,3,6-tetrahydropyridine, 1,2,3,4-tetrahydroquinoline, quinoxaline, quinazoline, pyridazine, cinnoline, 5,6,7,8-tetrahydroquinoxaline, 1,8-naphthyridine, and 4-azabenzimidazole rings. Some examples of unsaturated heterocyclic groups containing at least one sulfur atom include thiophene, thianaphthene, and benzothiophene rings. Some examples of unsaturated heterocyclic groups (R) containing at least one oxygen atom and at least one nitrogen atom include oxazole, isoxazole, benzoxazole, benzisoxazole, oxazoline, 1,2,5-oxadiazole (furazan), and 1,3,4-oxadiazole rings. Some examples of unsaturated heterocyclic groups (R) containing at least one nitrogen atom and at least one sulfur atom include thiazole, isothiazole, benzothiazole, benzoisothiazole, thiazoline, and 1,3,4-thiadiazole rings.

Unsaturated heterocyclic linkers (R) can be derived from any of the foregoing unsaturated heterocyclic groups by removal of one, two, or three hydrogen atoms from the unsaturated heterocyclic group to result in a divalent, trivalent, or tetravalent unsaturated heterocyclic linker, respectively.

I. Bifunctional Molecules with Binding Domain (R¹)

In a first aspect, the present disclosure is directed to compounds of the formula:

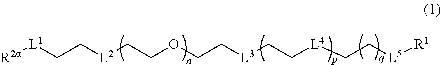

The variable R^{2a} in Formula (1) is a first reactive group that forms a linkage with a second reactive group. Some examples of R^{2a} first reactive groups include amine groups, thiol groups, carboxylic acid-containing groups, ester-containing groups, aldehyde-containing groups, amine-reactive groups, thiol-reactive groups, carboxylic acid-reactive groups, ester-reactive groups, aldehyde-reactive groups, click reaction reactive groups, and affinity binding groups. Some examples of amine-reactive groups include carboxy ester (i.e., “ester”) groups, including activated ester groups (e.g., N-hydroxysuccinimide group), as well as, for example, anhydride, aldehyde, carbodiimide, carbonate, isocyanate, haloalkyl, and epoxide groups. Some examples of thiol-reactive groups include maleimide, haloacetyl (e.g., iodoacetyl), vinylsulfone, and pyridyl disulfide groups. Some examples of carboxylic acid-reactive groups include primary amine, anhydride, and epoxide groups. Some examples of ester-reactive groups include primary amine, hydroxy, and epoxide groups. Some examples of aldehyde-reactive groups include primary amine, hydrazide, and alkoxyamine (amino-oxy) groups. Some examples of click reaction reactive groups include alkyne-containing, azide, thiol, maleimide, vinylsulfone, pyridyl disulfide, alkene-containing groups, and tetrazine-containing groups. In particular embodiments, R^{2a} is a click reaction reactive group, or more particularly, an alkyne-containing or azide-containing group. In particular embodiments, R^{2a} is an alkyne-containing group, or more particularly, a cycloalkyne group. The cycloalkyne group may be, for example, azadibenzocyclooctyne (DBCO), bicyclo[6.1.0]nonyne (BCN), or a difluorinated cyclooctyne (DIFO). All such reactive groups are well known in the art.

The compound of Formula (1) also includes several linkers, denoted as L¹, L², L³, L⁴, and L⁵, which are further defined as follows. The term “linker” indicates that the linkage being shown must have a bond on each side (i.e., is a diradical), whether or not a bond is depicted on either side of the linkage, and the linker can be inserted into Formula (1) in the orientation as shown or it may be flipped (e.g., CO(NH) may be inserted as NHCO, unless otherwise specified). The variable L¹ in Formula (1) is a bond, C(O), CH₂, C(O)NH, and C(O)O. In particular embodiments, L¹ is a bond or C(O). The variables L², L⁴, and L⁵ are independently selected from a bond, CH₂, C(O), NH, O, S, C(O)NH, C(O)O, OC(O)NH, OC(O)O, NHC(O)NH, and —OC(O)O—Ar—, or a CH₂ bound to any of the foregoing linkers, wherein Ar is an aromatic ring, such as any of those described above, and wherein an O atom can optionally be replaced with an S atom in any of the foregoing linkers. In particular embodiments, L² is C(O)O or C(O)NH, and/or L⁴ is OC(O)O—Ar, and/or L⁵ is C(O)NH, C(O)O, OC(O)NH, OC(O)O, or NHC(O)NH, or L⁵ is a linker in which CH₂ is bound to any of the foregoing linkers, or L⁵ is more particularly C(O)O or OC(O)O or a linker in which CH₂ is bound to any of the foregoing linkers. The variable L³ is selected from a bond, CH₂, CH₂CH₂, S—S, C(O)O, OC(O)O, C(O)NH, or OC(O)O—Ar, wherein Ar is an aromatic ring, and linked combinations of any two or more of the foregoing linkers (e.g., CH₂—S—S, CH₂CH₂—S—S, CH₂—C(O)O, CH₂CH₂—C(O)O, CH₂—OC(O)O—Ar, and CH₂CH₂—OC(O)O—Ar). In particular embodiments, L³ is S—S, CH₂—S—S, or CH₂CH₂—S—S. Notably, L¹ may be selected from any of the groups (include bonds) described above and combined with any selection of groups given above for L², and this combined with any selection of groups given above for L³, and this combined with any selection of groups given above for L⁴, and this combined with any selection of groups given above for L⁵, wherein it is herein understood that “groups” includes bonds. Moreover, any combination of L¹, L², L³, L⁴, and L⁵ can be further combined with any one or more groups provided above for R^{2a}. For example, in particular embodiments, R^{2a} is an alkyne-containing group (or cycloalkyne); L¹ is C(O); L² is C(O)O or C(O)NH; L³ is S—S; and L⁵ is selected from C(O)O and OC(O)O, or L⁵ is a linker in which CH₂ is bound to any of the foregoing linkers; or alternatively, R^{2a} is an azide-containing group; L¹ is C(O); L² is C(O)O or C(O)NH; L³ is S—S; and L⁵ is selected from C(O)O and OC(O)O, or L⁵ is a linker in which CH₂ is bound to any of the foregoing linkers.

The variable R¹ in Formula (1) is an aromatic or aliphatic ring or polycyclic ring system, wherein the aromatic or aliphatic ring or polycyclic ring system optionally contains one or more ring heteroatoms selected from N, O, and S, and the aromatic or aliphatic ring or polycyclic ring system optionally contains one or more non-H substituents selected from halogen atoms (e.g., F, Cl, or Br); oxo; linear or branched, cyclic or acyclic, saturated or unsaturated, halogenated or non-halogenated hydrocarbon groups containing 1-30 carbon atoms; NO₂; NR³R⁴; CN; C(O)R⁵; C(O)OR⁶; OC(O)R⁶; OR⁷; C(O)NR³R⁴; and NHC(O)R⁸. In the foregoing groups, R³, R⁴, R⁵, R⁶, R⁷, and R⁸ are independently selected from H and linear or branched alkyl groups containing 1-3 carbon atoms, such as any of those described above. In particular embodiments, R¹ is a group that is or includes a phenyl ring, or more particularly, a phenyl ring bearing precisely or at least one NO₂, halogen, methyl, methoxy, or CF₃ substituent. In other embodiments, R¹ is a group that is or includes a cyclopentyl or cyclohexyl ring, which is optionally substituted with one or more groups as provided above. Moreover, any group R¹ provided above may be selected and combined with any L⁵ linker described above to form an L⁵-R¹ moiety. In particular embodiments, L⁵ is C(O)O, C(O)OCH₂, or OC(O)O, which is attached to any of the R¹ groups described above. In alternative embodiments, L⁵ is a CR^{a}R^{b} linker, wherein R^{a} and R^{b} are independently selected from H; R⁹; OR⁹; NR¹⁰R¹¹; CN; C(O)R¹²; C(O)OR¹³; OC(O)R¹⁴; C(O)NR¹⁰R¹¹; and NHC(O)R¹⁵; wherein R⁹, R¹⁰, R¹¹, R¹², R¹³, R¹⁴, and R¹⁵ are independently selected from H and alkyl, alkenyl, and cycloalkyl groups containing 1-6 carbon atoms and optionally containing one or more halogen atoms. In other alternative embodiments, L⁵ is a bond or any of the groups provided above, and R¹ is optionally connected to L⁵ by a CR^{a}R^{b} linker, wherein R^{a} and R^{b} are independently selected from H; R⁹; OR⁹; NR¹⁰R¹¹; CN; C(O)R¹²; C(O)OR¹³; OC(O)R¹⁴, C(O)NR¹⁰R¹¹; and NHC(O)R¹⁵; wherein R⁹, R¹⁰, R¹¹, R¹², R¹³, R¹⁴, and R¹⁵ are independently selected from H and alkyl, alkenyl, and cycloalkyl groups containing 1-6 carbon atoms and optionally containing one or more halogen atoms. In some embodiments, precisely or at least one of R^{a} and R^{b} is selected from cyclopropyl, cyclobutyl, methoxy, CF₃, phenyl, cyclohexyl, cyclopentyl, or cyclopentadienyl.

In some embodiments, the L⁵-R¹ moiety corresponds to OC(O)—R^{1′}, wherein the R^{1′} group has any of the following structures, in which case L⁵ is selected as OC(O)O, OC(O)CH₂, or OC(O)CR^{a}R^{b}:

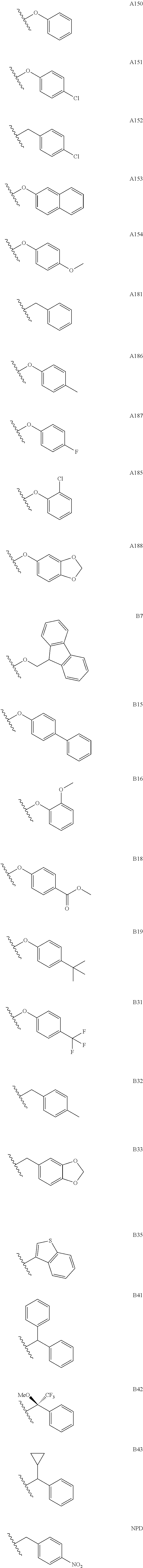

The variable n in Formula (1) corresponds to the number of ethylene oxide (EO) linker units shown in Formula (1). The variable n is an integer in the range of 0-100. In some embodiments, n is 0, in which case no EO linker units are present in the position shown in Formula (1). In other embodiments, n is precisely or at least 1, 2, 3, 4, 5, 6, 7, 8, 9, or 10 or a value within a range bounded by any two of the foregoing values (e.g., 1-10, 1-6, or 1-3), or a value within a range in which any of the foregoing values of n functions as a minimum and n=20, 30, 40, 50, 60, 70, 80, 90, or 100 functions as a maximum. In some embodiments, n is 0, 1, 2, or 3.

The variable p in Formula (1) corresponds to the number of CH₂CH₂L⁴ linker units shown in Formula (1). The variable p is an integer of 0-10. In some embodiments, p is 0, in which case no CH₂CH₂L⁴ linker units are present in Formula (1). In other embodiments, p is precisely or at least 1, 2, 3, 4, 5, 6, 7, 8, 9, or 10 or a value within a range bounded by any two of the foregoing values (e.g., 1-10, 1-6, 1-4, 1-3, or 1-2). In some embodiments p is 0, 1, or 2. Notably, any of the exemplary values of p provided above can be combined with any of the exemplary values of n provided above, and this can be further combined with any of the R^{2a}, L¹, L², L³, L⁴, L⁵, and R¹ possibilities provided above.

The variable q in Formula (1) corresponds to the number of CH₂ groups attached to the L⁵-R¹ moiety shown in Formula (1). The variable q is an integer of 0-50. In some embodiments, q is 0, in which case no CH₂ linker units connected to the L⁵-R¹ moiety are present in Formula (1), although L⁵ itself may or may not be a CH₂ or CR^{a}R^{b} linker. In other embodiments, q is precisely or at least 1, 2, 3, 4, 5, 6, 7, 8, 9, or 10 or a value within a range bounded by any two of the foregoing values (e.g., 1-10, 1-6, 1-3, or 1-2), or a value within a range in which any of the foregoing values of n functions as a minimum and n=20, 30, 40, or 50 functions as a maximum. In some embodiments, q is 0, 1, 2, or 3. Notably, any of the exemplary values of q provided above can be combined with any of the exemplary values of n and p provided above, and this can be further combined with any of the R^{2a}, L¹, L², L³, L⁴, L⁵, and R¹ possibilities provided above.

II. Bifunctional Molecules with Cell Penetration Moiety (R³)

In a second aspect, the present disclosure is directed to compounds of the formula:

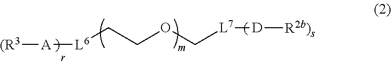

The variable R^{2b} in Formula (2) is a second reactive group that forms a linkage with a first reactive group, wherein the first reactive group may be any of the reactive groups provided as R^{2a}, as described above under Formula (1). R^{2b} may be selected from any of the same groups as provided above for R^{2a}. Some examples of R^{2b} second reactive groups include amine groups, thiol groups, carboxylic acid-containing groups, ester-containing groups, aldehyde-containing groups, amine-reactive groups, thiol-reactive groups, carboxylic acid-reactive groups, ester-reactive groups, aldehyde-reactive groups, click reaction reactive groups, and affinity binding groups. Some examples of amine-reactive groups include carboxy ester (i.e., “ester”) groups, including activated ester groups (e.g., N-hydroxysuccinimide group), as well as, for example, anhydride, aldehyde, carbodiimide, carbonate, isocyanate, haloalkyl, and epoxide groups. Some examples of thiol-reactive groups include maleimide, haloacetyl (e.g., iodoacetyl), vinylsulfone, and pyridyl disulfide groups. Some examples of carboxylic acid-reactive groups include primary amine, anhydride, and epoxide groups. Some examples of ester-reactive groups include primary amine, hydroxy, and epoxide groups. Some examples of aldehyde-reactive groups include primary amine, hydrazide, and alkoxyamine (amino-oxy) groups. Some examples of click reaction reactive groups include alkyne-containing, azide, thiol, maleimide, vinylsulfone, pyridyl disulfide, alkene-containing groups, and tetrazine-containing groups. In particular embodiments, R^{2b} is a click reaction reactive group, or more particularly, an alkyne-containing or azide-containing group. In particular embodiments, R^{2b} is an azide-containing group. In the case where R^{2b} is an alkyne-containing group, the alkyne-containing group may be a cycloalkyne group, such as, for example, azadibenzocyclooctyne (DBCO), bicyclo[6.1.0]nonyne (BCN), or a difluorinated cyclooctyne (DIFO). All such reactive groups are well known in the art.

The compound of Formula (2) also includes two linkers, denoted as L⁶ and L⁷, which are further defined as follows. The term “linker” indicates that the linkage being shown must have a bond on each side (i.e., is a diradical), whether or not a bond is depicted on either side of the linkage, and the linker can be inserted into Formula (2) in the orientation as shown or it may be flipped (e.g., CO(NH) may be inserted as NHCO, unless otherwise specified).

The subscript r in Formula (2) represents the number of R³-A arms on the linker L⁶, wherein r is selected from an integer in the range of 1-6. The subscript s in Formula (2) represents the number of D-R^{2b} arms on the linker L⁷, wherein s is selected from an integer in the range of 1-6. The subscripts r and s may be independently selected. In some embodiments, r is 1, 2, 3, 4, 5, or 6 (or a range therein) and s is 1. In other embodiments, s is 1, 2, 3, 4, 5, or 6 (or a range therein) and r is 1. In some embodiments, r is 2-6 and s is 1. In other embodiments, r is 2 or 3 and s is 1. In other embodiments, r is 1 and s is 2-6. In other embodiments, r is 1 and s is 2 or 3.

The variable L⁶ in Formula (2) is either: (i) a non-branching linker selected from CH₂, C(O), NH, O, S, C(O)NH, C(O)O, OC(O)NH, OC(O)O, and NHC(O)NH, or one or more of these attached together, wherein r is 1, which corresponds to a single R³-A arm on L⁶; or (ii) a branching linker selected from C, CH, N, B, or a linear or branched, cyclic or acyclic, saturated or unsaturated hydrocarbon linker group Re containing 2-50 carbon atoms on which an r number (i.e., 2, 3, 4, 5, or 6) of R³-A arms are attached, wherein Re optionally contains one or more heteroatoms selected from N, O, and S; or (iii) one or more non-branching linkers in (i) attached to a branching linker (ii). In the event that L⁶ is C, three (r=3) R³-A arms can be attached, which results in the (R³-A)ᵣ-L⁶-moiety being (R³-A)₃C—. In the event that L⁶ is CH, two (r=2) R³-A arms can be attached, which results in the (R³-A)ᵣ-L⁶-moiety being (R³-A) 2CH—. Similarly, in the event that L⁶ is N or B, two (r=2) R³-A arms can be attached, which results in the (R³-A)ᵣ-L⁶-moiety being (R³-A)₂N— or (R³-A)₂B—. As another example, in the event that L⁶ is a phenylene ring (denoted as —C₆H₄—), two, three, or four R³-A arms can be attached, which results in the (R³-A)ᵣ-L⁶-moiety being, respectively, (R³-A) 2-C₆H₂—, (R³-A)₃-C₆H—, or (R³-A) 4-C₆—. As another example, in the event that L⁶ is —CH(CH₂—)₂—, two (r=2) R³-A arms can be attached, which results in the (R³-A)ᵣ-L⁶-moiety being [(R³-A) CH_{2] 2}CH—; or four (r=4) R³-A arms can be attached, which results in the (R³-A)ᵣ-L⁶-moiety being [(R³-A)₂CH] 2CH—; or six (r=6) R³-A arms can be attached, which results in the (R³-A)ᵣ-L⁶-moiety being [(R³-A)₃C]₂CH—. As another example, in the event that L⁶ is —C(CH₂CH₂—)₃—, three (r=3) R³-A arms can be attached, which results in the (R³-A)ᵣ-L⁶-moiety being [(R³-A)CH₂CH_{2] 3}C—. As another example, in the event that L⁶ is —C(CH₂CH₂OCH₂CH₂OCH₂CH₂—)³—, three (r=3) R³-A arms can be attached, which results in the (R³-A)ᵣ-L⁶-moiety being [(R³-A) CH₂CH₂OCH₂CH₂OCH₂CH_{2] 3}C—. In some embodiments, L⁶ may alternatively be a bond, in which case r is 1.

The variable L⁷ in Formula (2) is either: (i) a non-branching linker selected from CH₂, C(O), NH, O, S, C(O)NH, C(O)O, OC(O)NH, OC(O)O, and NHC(O)NH, or one or more of these attached together, wherein s is 1, which corresponds to a single D-R^{2b} arm on L⁷; or (ii) a branching linker selected from C, CH, N, B, or a linear or branched, cyclic or acyclic, saturated or unsaturated hydrocarbon linker group Re containing 2-50 carbon atoms on which an s number (i.e., 2, 3, 4, 5, or 6) of D-R^{2b} arms are attached, wherein R^{c} optionally contains one or more heteroatoms selected from N, O, and S, or (iii) one or more non-branching linkers in (i) attached to a branching linker (ii). In the event that L⁷ is C, three (s=3) D-R^{2b} arms can be attached, which results in the -L⁷-(D-R^{2b}) s moiety being —C(D-R^{2b}) 3. In the event that L′ is CH, two (s=2) D-R^{2b} arms can be attached, which results in the -L⁷-(D-R^{2b}) s moiety being-CH (D-R^{2b}) 2. Similarly, in the event that L⁷ is N or B, two (s=2) D-R^{2b} arms can be attached, which results in the -L⁷-(D-R^{2b}) s moiety being-N (D-R^{2b}) 2 or —B (D-R^{2b}) 2. As another example, in the event that L⁷ is a phenylene ring (denoted as —C₆H₄—), two, three, or four D-R^{2b} arms can be attached, which results in the -L⁷-(D-R^{2b}) s moiety being, respectively, —C₆H₂-(D-R^{2b}) 2, —C₆H-(D-R^{2b}) 3, or —C₆-(D-R^{2b}) 4. As another example, in the event that L⁷ is —CH(CH₂—)₂—, two (s=2) D-R^{2b} arms can be attached, which results in the -L⁷-(D-R^{2b}) s moiety being-CH [CH₂ (D-R^{2b})] 2; or four (s=4) D-R^{2b} arms can be attached, which results in the -L⁷-(D-R^{2b}) s moiety being-CH [CH (D-R^{2b}) 2] 2; or six (s=6) R³-A arms can be attached, which results in the -L⁷-(D-R^{2b}) s moiety being-CH [C (D-R^{2b}) 3] 2. As another example, in the event that L⁷ is —C(CH₂CH₂—)³—, three (s=3) D-R^{2b} arms can be attached, which results in the -L⁷-(D-R^{2b}) s moiety being-C(CH₂CH₂-D-R^{2b}) 3. As another example, in the event that L⁷ is —C(CH₂CH₂OCH₂CH₂OCH₂CH₂—)³—, three (s=3) D-R^{2b} arms can be attached, which results in the -L⁷-(D-R^{2b}) s moiety being-C(CH₂CH₂OCH₂CH₂OCH₂CH₂-D-R^{2b}) 3. In some embodiments, L⁷ may alternatively be a bond, in which case s is 1.

The variables A and D in Formula (2) are independently selected from a bond or a hydrocarbon linker containing 1-50 carbon atoms and optionally containing one or more heteroatoms selected from halogen atoms (e.g., F, Cl, or Br), N, O, and S. In other embodiments, the number of carbon atoms in A and D may independently selected from 1-40, 1-30, 1-20, 1-12, 1-8, 1-6, 1-5, 1-4, 1-3, 2-50, 2-40, 2-30, 2-20, 2-12, 2-8, 2-6, 2-5, 2-4, or 2-3 carbon atoms. In the event that one or more N, O, or S heteroatoms is present, they may be included in one or more non-H substituents, such as, for example, oxo; NO₂; NR³R⁴; CN; C(O)R⁵; C(O)OR⁶; OC(O)R⁶; OR⁷; C(O)NR³R⁴; and NHC(O)R⁸. In the foregoing groups, R³, R⁴, R⁵, R⁶, R⁷, and R⁸ are independently selected from H and linear or branched alkyl groups containing 1-3 carbon atoms, such as any of those described above. In some embodiments, A is a bond while D is a hydrocarbon linker containing 1-50 carbon atoms. In other embodiments, D is a bond while A is a hydrocarbon linker containing 1-50 carbon atoms. In other embodiments, both A and D are bonds. In other embodiments, both A and D are independently selected from hydrocarbon linker containing 1-50 carbon atoms. In some embodiments, at least one (or both) of A and D contains a polyalkylene oxide segment, which may be of the formula —(CHR^{a}CHR^{b}O)ᵥ—, wherein R^{a} and R^{b} are typically independently selected from H and CH₃, or more typically, both R^{a} and R^{b} are H or one of R^{a} and R^{b} may be CH₃, and wherein v may be precisely, at least, or greater than 1, 2, 3, 4, 5, or 6.

The variable R³ in Formula (2) is independently a linear or branched, cyclic or acyclic, saturated or unsaturated hydrocarbon group containing 4-30 (or more particularly, 5-30, 6-30, 8-30, 10-30, or 12-30) carbon atoms optionally containing one or more heteroatoms selected from N, O, S, and B. Such hydrocarbon groups have been discussed in detail earlier above, and R³ may be selected from among any such hydrocarbon groups. The variable R³ functions as a cell penetration moiety, and thus, should be substantially hydrophobic. In one set of embodiments, R³ is a saturated or unsaturated fatty acyl group. Some examples of saturated fatty acyl groups include butyryl, capryloyl, caproyl, lauroyl, myristoyl, palmitoyl, stearoyl, arachidoyl, behenoyl, lignoceroyl, and cerotoyl. Some examples of unsaturated fatty acyl groups include oleoyl, palmitoleoyl, myristoleoyl, erucoyl, linoleoyl, and arachidonyl. In another set of embodiments, R³ is a sterol or steroid group, or more particularly, cholesterol or a derivative thereof. The derivative of cholesterol preserves the four ring fused system of cholesterol but may have the OH group or alkene bond removed, changed in position, altered (e.g., OH may be esterified), or one or more additional groups (e.g., OH or double bond) may be included. Some examples of cholesterol derivatives include 20-hydroxycholesterol, 22-hydroxycholesterol, 24-hydroxycholesterol, 25-hydroxycholesterol, 27-hydroxycholesterol, 7α-hydroxycholesterol, 7β-hydroxycholesterol, 7-ketocholesterol, 5,6α-epoxycholesterol, 5,6β-epoxycholesterol, lanosterol, dihydrolanosterol, lathosterol, zymosterol, Δ7-cholesterol, 48-cholesterol, Δ8,14-dimethylsterol, Δ5,7-cholestadienol, and Δ8,24-dimethylsterol. In another set of embodiments, R³ is a non-steroid saturated or unsaturated polycyclic group, e.g., decalinyl, naphthyl, phenanthryl, anthracenyl, fluorenyl, pyrenyl, chrysenyl and benzo or dibenzo derivatives of any of these, or a hydrocarbon linking group attached to any of these.

The variable m in Formula (2) corresponds to the number of ethylene oxide (EO) linker units shown in Formula (2). The variable m is an integer in the range of 0-100. In some embodiments, m is 0, in which case no EO linker units are present in the position shown in Formula (2). In other embodiments, m is precisely or at least 1, 2, 3, 4, 5, 6, 7, 8, 9, or 10 or a value within a range bounded by any two of the foregoing values (e.g., 1-10, 1-6, or 1-3), or a value within a range in which any of the foregoing values of m functions as a minimum and m=20, 30, 40, 50, 60, 70, 80, 90, or 100 functions as a maximum. In some embodiments, m is 0, 1, 2, or 3.

Notably, any two or more of the variables R^{2b}, R³, A, D, L⁶, L⁷, r, m, and s may be specifically and independently selected from those provided above and combined to arrive at one or more specific types of molecules of Formula (2). For example, in one set of embodiments, R^{2b} is an azide (or alkyne); r is 1, 2 or 3; sis 1; R³ is a saturated or unsaturated fatty acyl group; and m is 1-10. Alternatively, in another set of embodiments, R^{2b} is an azide (or alkyne); r is 1, 2 or 3; s is 1; R³ is cholesterol (or steroid or sterol) or a derivative thereof; and m is 1-10. Alternatively, in another set of embodiments, R^{2b} is an azide (or alkyne); r is 1; sis 1, 2 or 3; R³ is a saturated or unsaturated fatty acyl group; and m is 1-10. Alternatively, in another set of embodiments, R^{2b} is an azide (or alkyne); r is 1; s is 1, 2 or 3; R³ is cholesterol (or steroid or sterol) or a derivative thereof; and m is 1-10.

In some embodiments, r is 1, which results in the following formula:

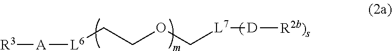

In other embodiments, s is 1, which results in the following formula:

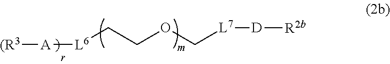

In other embodiments, r is 1 and s is 1, which results in the following formula:

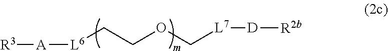

III. Full Molecules with Cell Penetration Moiety (R³) and Binding Domain (R¹)

In a third aspect, the present disclosure is directed to compounds that combine the molecules of Formulas (1) and (2) into one molecule by reaction of the R^{2a} and R^{2b} reactive groups, wherein the compounds have the following formula:

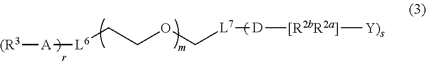

wherein Y=

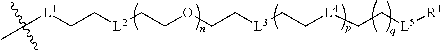

In Formula (3) above, R¹, R³, A, D, L¹, L², L³, L⁴, L⁵, L⁶, L⁷, r, m, s, n, p, and q are as defined above under Formula (1) and (2), and each of these variables may be independently selected from any of the groups provided above to arrive at one or more specific types of molecules of Formula (3). The linking group denoted as [R^{2b}R^{2a}] represents a reaction product between R^{2b} and R^{2a}. [R^{2b}R^{2a}] may represent a reaction product between, for example, click reaction groups; amine and amine-reactive groups; thiol and thiol-reactive groups; carboxylic acid-containing groups and carboxylic acid-reactive groups; ester-containing groups and ester-reactive groups; or aldehyde-containing groups and aldehyde-reactive groups. In particular embodiments, [R^{2b}R^{2a}] comprises a click reaction product, such as a 1,2,3-triazole. In separate or further embodiments, r is 1 or 2-6 and sis 1; or r is 1 and sis 1 or 2-6. In separate or further embodiments, at least one (or both) of A and D contains a polyalkylene oxide segment. In separate or further embodiments, m is 1-10. In a first set of embodiments, [R^{2b}R^{2a}] comprises a 1,2,3-triazole; L¹ is C(O); L² is C(O)O or C(O)NH; L³ is S—S; and L⁵ is selected from C(O)O and OC(O)O, or L⁵ is a linker in which CH₂ is bound to any of the foregoing linkers (with all remaining variables in Formula (3) defined and independently selected as provided earlier above). In a second set of embodiments, [R^{2b}R^{2a}] comprises a 1,2,3-triazole; r is 2 or 3; s is 1; R³ is a saturated or unsaturated fatty acyl group; and m is 1-10 (with all remaining variables in Formula (3) defined and independently selected as provided earlier above). In a third set of embodiments, [R^{2b}R^{2a}] comprises a 1,2,3-triazole; r is 2 or 3; s is 1; R³ is cholesterol (or steroid or sterol) or a derivative thereof; and m is 1-10 (with all remaining variables in Formula (3) defined and independently selected as provided earlier above). In a fourth set of embodiments, [R^{2b}R^{2a}] comprises a 1,2,3-triazole; r is 1; s is 2 or 3; R³ is a saturated or unsaturated fatty acyl group; and m is 1-10 (with all remaining variables in Formula (3) defined and independently selected as provided earlier above). In a fifth set of embodiments, [R^{2b}R^{2a}] comprises a 1,2,3-triazole; r is 1; s is 2 or 3; R³ is cholesterol (or steroid or sterol) or a derivative thereof; and m is 1-10 (with all remaining variables in Formula (3) defined and independently selected as provided earlier above).

IV. Conjugates of Payload (P) with Molecules of Formula (3)

In a fourth aspect, the present disclosure is directed to conjugates of payload (P) with molecules of Formula (3) containing cell penetration moiety (R³) and binding domain (R¹), wherein the compounds have the following formula:

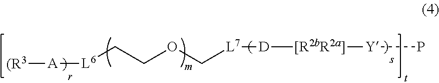

wherein Y′ is a linkage having the following structure:

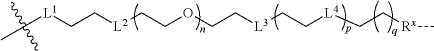

In Formula (4) above, R³, A, D, L¹, L², L³, L⁴, L⁶, L⁷, r, m, s, n, p, and q are as defined or exemplified above under Formulas (1), (2), and (3). Each of the foregoing variables may be independently selected from among the possibilities provided above and combined. The linking group denoted as [R^{2b}R^{2a}] represents a reaction product between R^{2b} and R^{2a}, as described above under Formula (3). [R^{2b}R^{2a}] can be any of the reaction products, including click reaction products, as described in detail under Formula (3). R^{x} is a linkage reaction product between L⁵-R¹, as defined under Formula (1), and a group (e.g., amine or thiol) or domain on the P (payload) molecule. The bonding between L⁵-R¹ and P (i.e., as the bond R^{x}—P) may be covalent or non-covalent (e.g., ionic or hydrogen bond). In Formula (4), the dashed bond between R^{x} and P indicates a covalent or non-covalent bond. The variable t represents the number of molecules of Formula (3) bonded to P. The variable t is at least 1 and may more particularly be precisely or at least, for example, 1, 2, 3, 4, 5, 6, 7, 8, 9, or 10.

The variable P in Formula (4) is a peptide-containing or nucleic acid-containing molecule. In the case where P is a peptide-containing molecule, P may be a peptide (typically up to less than 50 amino acid units) or protein (typically at least or greater than 50 or at least or above 75, 100, 200, 500, or 1000 amino acid units). Alternatively, the peptide or protein has a molecular weight of 10-500 kDa, or more particularly, a molecular weight of 30-300 kDa, 50-300 kDa, 30-250 kDa, 50-250 kDa, 30-200 kDa, 50-200 kDa, 30-150 kDa, or 50-150 kDa. In the case where P is nucleic acid-containing molecule, P may be an oligonucleotide (typically about 13-25 nucleotides) or a nucleic acid (typically greater than 25 or at least or above 30, 50, 75, 100, 200, 500, or 1000 nucleotides), wherein the oligonucleotide or nucleic acid may be DNA or RNA or a particular form thereof (e.g., mRNA). In some embodiments, one of or both of A and D contains a polyalkylene oxide segment, as described earlier above. In further or separate embodiments, m is a value of 1-10.

In one set of embodiments of Formula (4), [R^{2b}R^{2a}] comprises a click reaction product; L¹ is C(O); L² is C(O)O or C(O)NH; L³ is S—S; and L⁵ is selected from C(O)O and OC(O)O, or L⁵ is a linker in which CH₂ is bound to any of the foregoing linkers. In another set of embodiments of Formula (4), [R^{2b}R^{2a}] comprises a click reaction product; r is 2 or 3; s is 1; R³ is a saturated or unsaturated fatty acyl group; and m is 1-10. In another set of embodiments of Formula (4), [R^{2b}R^{2a}] comprises a click reaction product; r is 2 or 3; s is 1; R³ is cholesterol or a derivative thereof; and m is 1-10. In another set of embodiments of Formula (4), [R^{2b}R^{2a}] comprises a click reaction product; r is 1; s is 2 or 3; R³ is a saturated or unsaturated fatty acyl group; and m is 1-10. In another set of embodiments of Formula (4), [R^{2b}R^{2a}] comprises a click reaction product; r is 1; s is 2 or 3; R³ is cholesterol or a derivative thereof; and m is 1-10. Moreover, each of R¹, R³, A, D, L¹, L², L³, L⁴, L⁵, L⁶, L⁷, [R^{2b}R^{2a}], r, m, s, n, p, q, and t may be independently selected from among the possibilities provided anywhere in this disclosure and combined to form a molecule of Formula (4).

V. Conjugates of Payload (P) with Molecules of Formula (1)

In a fifth aspect, the present disclosure is directed to conjugates of payload (P) with molecules of Formula (1) to form compounds having the following formula:

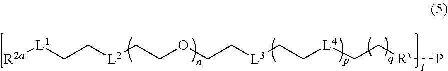

In Formula (5) above, R^{2a}, L¹, L², L³, L⁴, n, p, q, R^{x}, and P are as defined or exemplified above under Formulas (1), (3), and (4). Each of the foregoing variables may be independently selected from among the possibilities provided anywhere in this disclosure and combined. In Formula (5), the dashed bond between R^{x} and P indicates a covalent or non-covalent bond, as described under Formula (4). The variable t represents the number of molecules of Formula (1) bonded to P. The variable t is at least 1 and may more particularly be precisely or at least, for example, 1, 2, 3, 4, 5, 6, 7, 8, 9, or 10.

VI. Methods of Producing Full Molecules of Formula (3) Containing Cell Penetration Moiety (R³) and Binding Domain (R¹)

The compounds of Formula (3) and sub-formulas thereof can be synthesized by methods well known in the art. In particular embodiments, the compounds of Formula (3) are produced by reacting a molecule of Formula (1) with a molecule of Formula (2) under conditions (as well known in the art) where reactive groups R^{2a} and R^{2b} react and form a linkage.

More particularly, the method involves reacting a molecule of the following formula:

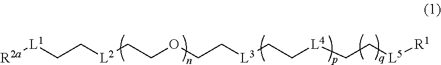

with a molecule of the following formula:

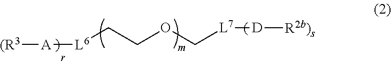

under conditions in which R^{2a} forms a linkage with R^{2b} to result in the following molecule:

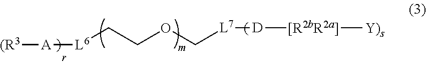

wherein Y=

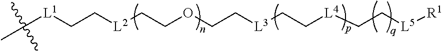

In the above method, R¹, R³, A, D, L¹, L², L³, L⁴, L⁵, L⁶, L⁷, r, m, s, n, p, q, and [R^{2b}R^{2a}] are as defined or exemplified Formulas (1), (2), and (3). Each of the foregoing variables may be independently selected from among the possibilities provided anywhere in this disclosure and combined. In various embodiments, [R^{2b}R^{2a}] represents a reaction product between click reaction groups; amine and amine-reactive groups; thiol and thiol-reactive groups; carboxylic acid-containing groups and carboxylic acid-reactive groups; ester-containing groups and ester-reactive groups; or aldehyde-containing groups and aldehyde-reactive groups. In particular embodiments, [R^{2b}R^{2a}] represents a click reaction product, such as a 1,2,3-triazole. The conditions under which R^{2a} forms a linkage with R^{2b} to form a reaction product [R^{2b}R^{2a}] are well known in the art.

### VII. Methods of Producing Conjugate of Formula (4) Containing Full Molecules of Formula (3) Bound to Payload P, by Either a One-Step or Two-Step Approach

### One-Step Approach

For a one-step approach, the following methodology may be used:

reacting a payload molecule (P) with a molecule of the following formula:

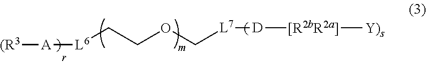

wherein Y=

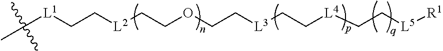

under conditions in which R¹ reacts with a group in P to result in the following molecule:

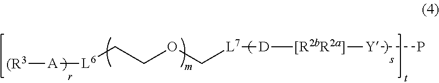

wherein Y′ is a linkage having the following structure:

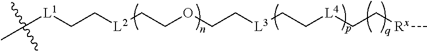

In the above one-step approach, R¹, R³, A, D, L¹, L², L³, L⁴, L⁵, L⁶, L⁷, r, m, s, n, p, q, t, [R^{2b}R^{2a}], R^{x}, and P are as defined or exemplified under Formulas (1), (2), (3), and (4). Each of the foregoing variables may be independently selected from among the possibilities provided anywhere in this disclosure and combined. The conditions under which R¹ reacts with a group in P are well known in the art. The one-step approach is a more convenient conjugation process for the delivered payload P which has a high effective covalent chemical linkage or strong non-covalent binding activity with the whole STEP molecule under Formula (4).

### Two-Step Approach

For a two-step approach, the following methodology may be used:

in a first step, reacting a payload molecule (P) with a molecule of the following formula:

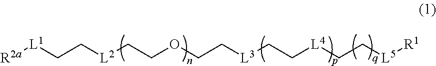

under conditions in which R¹ reacts with a group or domain in P to result in the following molecule:

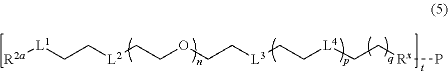

and in a second step, reacting the conjugate molecule of Formula (5) with a molecule of the Formula (2) having the following structure:

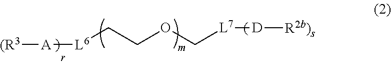

under conditions in which R^{2a} (a second reactive group) forms a linkage with R^{2b} to result in the following molecule of Formula (4):

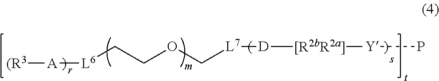

wherein Y′ is a linkage having the following structure:

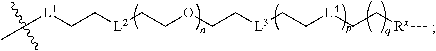

In the above two-step approach, R¹, R^{2a}, R³, A, D, L¹, L², L³, L⁴, L⁵, L⁶, L⁷, r, m, s, n, p, q, t, [R^{2b}R^{2a}], R^{x}, and P are as defined or exemplified under Formulas (1), (2), (3), and (4). Each of the foregoing variables may be independently selected from among the possibilities provided anywhere in this disclosure and combined. The conditions under which R¹ reacts with a group in P are well known in the art. The two-step approach provides a more effective covalent chemical linkage or non-covalent binding of the payload P to the molecules under Formula (1) due to lack of chemical structure hindrance of the cell penetrating molecules under Formula (2). The two-step approach is a suitable conjugation process for the delivered payload P which has no effective covalent chemical linkage or weak non-covalent binding activity with the whole STEP molecule under Formula (4).

### VIII. Method of Producing Conjugates of Formula (5) Containing Molecule(s) of Formula (1) Bound to Payload (P)

To produce conjugates of Formula (5) containing molecule(s) of Formula (1) bound to payload (P), the following methodology may be used:

reacting a payload molecule (P) with a molecule of the following formula:

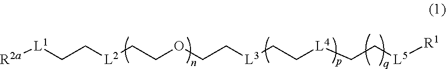

under conditions in which R¹ reacts with a group in P to result in the following molecule:

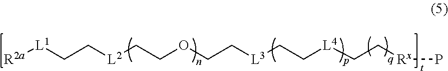

In the above method, R¹, R^{2a}, L¹, L², L³, L⁴, L⁵, n, p, q, t, R^{x}, and P are as defined or exemplified above under Formulas (1), (3), (4), and (5). Each of the foregoing variables may be independently selected from among the possibilities provided anywhere in this disclosure and combined. In Formula (5), the dashed bond between R^{x} and P indicates a covalent or non-covalent bond, as described under Formula (4). The conditions under which R¹ reacts with a group in P are well known in the art. Reactions that result in covalent bonding of R¹ to P (i.e., “covalent bond reaction”) are suitable for payloads P which have reactive chemical groups including but not limited to —NH₂, —COOH, —OH, —SH, and —N₃. Reactions that result in non-covalent bonding of R¹ to P (i.e., “non-covalent bond reaction”) are suitable for payloads P which contain groups that provide strong non-covalent interactions, such as hydrogen bond, π-π stacking, and ion-ion interactions.

### IX. Method of Delivering a Payload (P) to a Cell by Contacting the Conjugate of Formula (4) to a Cell

To deliver a payload (P) to a cell, a conjugate molecule of the Formula (4) is contacted or interacted with a cell with resulting penetration of the conjugate molecule of the Formula (4) into the cell by means of the cell penetration moiety R³, wherein the conjugate molecule of Formula (4) has the following formula:

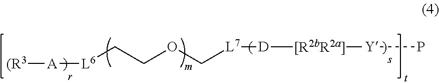

wherein Y′ is a linkage having the following structure:

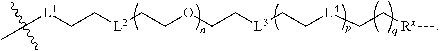

In Formula (4) above, R³, A, D, L¹, L², L³, L⁴, L⁶, L⁷, r, m, s, n, p, q, t, and P are as defined or exemplified above under Formulas (1), (2), (3), and (4). Each of the foregoing variables may be independently selected from among the possibilities provided anywhere in this disclosure and combined. The linking group denoted as [R^{2b}R^{2a}] represents a reaction product between R^{2b} and R^{2a}, as described above under Formula (3). [R^{2b}R^{2a}] can be any of the reaction products, including click reaction products, as described in detail under Formula (3). R^{x} is a linkage reaction product between L⁵-R¹, as defined under Formula (1), and a group (e.g., amine or thiol) or domain on the P (payload) molecule. The bonding between L⁵-R¹ and P (i.e., as the bond R^{x}—P) may be covalent or non-covalent (e.g., ionic or hydrogen bond). In Formula (4), the dashed bond between R^{x} and P indicates a covalent or non-covalent bond. The variable t represents the number of molecules of Formula (3) bonded to P. The variable t is at least 1 and may more particularly be precisely or at least, for example, 1, 2, 3, 4, 5, 6, 7, 8, 9, or 10.

### X. Pharmaceutically Acceptable Salts

Any of the molecules according to formulas and sub-formulas disclosed herein may be in the form of a pharmaceutically acceptable salt. As used herein, the term “pharmaceutically acceptable salt” includes both acid and base addition salts, wherein the compound is modified by making acid or base salts thereof. As the molecules described herein may include one or more amino groups or linkers (e.g., —NH—), acid addition salts are particularly considered. Examples of pharmaceutically acceptable salts include but are not limited to mineral or organic acid salts of basic residues such as amines. Pharmaceutically acceptable salts include conventional non-toxic salts or quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids, such as acetic, propionic, succinic, glycolic, stearic, lactic, maleic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, naphthalene sulfonic, methanesulfonic, ethane disulfonic, and oxalic acids. The pharmaceutically acceptable salts of a compound disclosed herein can be synthesized from the parent compound, which contains a basic or acidic moiety, by conventional chemical methods. Suitable salts include those described in P. Heinrich Stahl, Camille G. Wermuth (Eds.), Handbook of Pharmaceutical Salts Properties, Selection, and Use, 2002.

### XI. Pharmaceutical Compositions

In another aspect, the present disclosure is directed to pharmaceutical compositions of any of the conjugates described above. In the pharmaceutical composition, any of the conjugates described above, such as depicted in Formulas (4) or (5) or sub-formula thereof, is dispersed in a pharmaceutically acceptable carrier, i.e., vehicle or excipient. The compound is dispersed in the pharmaceutically acceptable carrier by either being mixed (e.g., in solid form with a solid carrier) or dissolved or emulsified in a liquid carrier. The pharmaceutical composition may or may not also be formulated together with one or more additional active ingredients or adjuvants (i.e., additives) that improve the overall efficacy of the pharmaceutical composition, particularly as relates to the treatment of a disease or condition by the payload (P) being delivered in to cells, wherein P may be a peptide-containing or nucleic acid-containing molecule, as described in detail earlier above.

The phrase “pharmaceutically acceptable” refers herein to those compounds, materials, compositions (e.g., acids or bases), and/or dosage forms which are, within the scope of sound medical judgment, suitable for administration to a subject. The phrase “pharmaceutically acceptable carrier,” as used herein, refers to a pharmaceutically acceptable vehicle, such as a liquid or solid filler, diluent, carrier, manufacturing aid (e.g., lubricant, talc magnesium, calcium or zinc stearate, or stearic acid), solvent, or encapsulating material, which serves to carry the therapeutic composition for administration to the subject. Each carrier should be “acceptable” in the sense of being compatible with the other ingredients of the formulation and physiologically safe to the subject. Any of the carriers known in the art can be suitable herein depending on the mode of administration.

Some examples of materials that can serve as pharmaceutically-acceptable carriers include water; isotonic saline; pH buffering agents; and sugars (e.g., lactose, glucose, sucrose, and oligosaccharides, such as sucrose, trehalose, lactose, or dextran). Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

Any of the conjugates described above may be formulated into pharmaceutical compositions and dosage forms according to methods well known in the art. The pharmaceutical compositions of the present invention may be specially formulated for administration in liquid or solid form. In some embodiments, the pharmaceutical formulation is formulated for oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); topical application (e.g., as a cream, ointment, or spray); intravaginal or intrarectal administration (e.g., as a pessary, cream or foam); sublingual or buccal administration; ocular administration; transdermal administration; or nasal administration. In some embodiments, the pharmaceutical composition is a liquid formulation designed for administration by injection.

The conjugate can be incorporated in a pharmaceutical composition suitable for use as a medicament, which may be for human or animal use. The pharmaceutical compositions may be, for example, in an injectable formulation, a liquid, cream or lotion for topical application, an aerosol, a powder, granules, tablets, suppositories or capsules, such as for instance, enteric coated capsules, or the like. The pharmaceutical compositions may also be delivered in or on a lipid formulation, such as, for example, an emulsion or a liposome preparation. The pharmaceutical compositions are preferably sterile, non-pyrogenic and isotonic preparations, optionally with one or more of the pharmaceutically acceptable additives listed below. Pharmaceutical compositions of the molecule are preferably stable compositions which may comprise one or more of the following: a stabilizer, a surfactant, such as a nonionic surfactant, and optionally a salt and/or a buffering agent. The pharmaceutical composition may be in the form of an aqueous solution, or in a lyophilized form. The stabilizer may, for example, be an amino acid, such as, for example, glycine; or an oligosaccharide, such as for example, sucrose, tetralose, lactose, or a dextran. Alternatively, the stabilizer may be a sugar alcohol, such as, for example, mannitol; or a combination thereof. In embodiments, the stabilizer or combination of stabilizers constitutes from about 0.1% to about 10% weight for weight of the molecule. The surfactant is typically a nonionic surfactant, such as a polysorbate. Some examples of suitable surfactants include Tween® 20, Tween® 80; a polyethylene glycol or a polyoxyethylene polyoxypropylene glycol, such as Pluronic™ F-68 at from about 0.001% (w/v) to about 10% (w/v). The salt or buffering agent may be any salt or buffering agent, such as, for example, sodium chloride, or sodium/potassium phosphate, respectively. In embodiments, the buffering agent maintains the pH of the pharmaceutical composition in the range of about 5 to 8 or about 6 to 7. The salt and/or buffering agent is also useful to maintain the osmolality at a level suitable for administration to a human or an animal. Preferably, the salt or buffering agent is present at a roughly isotonic concentration of about 150 mM to about 300 mM.

The pharmaceutical composition containing the conjugate may additionally contain one or more conventional additives. Some examples of such additives include solubilizers, such as, for example, PEG or lipid-PEG, such as, cholesterol-PEG500, PEG1000-N₃, PEG2000-N₃, cholesterol-PEG1000, cholesterol-PEG1500, cholesterol-PEG2000, cholesterol-PEG3000, cholesterol-PEG5000; glycerol; antioxidants, such as, for example, benzalkonium chloride (a mixture of quaternary ammonium compounds, known as “quats”), benzyl alcohol, chloretone or chlorobutanol; anesthetic agents, such as, for example, morphine derivatives; and isotonic agents, such as described above. As a further precaution against oxidation or other spoilage, the pharmaceutical compositions may be stored under nitrogen or argon gas in vials sealed with impermeable stoppers. In some embodiments, any one or more of the above listed additives may be excluded from the pharmaceutical composition.

### XII. Methods of Delivery and Treatment

In other aspects, the present disclosure is directed to methods of delivering a payload (P) to a subject and for treating a disease or condition in a subject by such delivery. Typically, the payload is a peptide-containing or nucleic acid-containing payload molecule, such as any of those described in detail earlier above. The delivery may be achieved by any of the administration modes provided earlier above, such as by injection or oral intake. In some embodiments, the conjugate is delivered alone, while in other embodiments the conjugate is delivered in the form of a pharmaceutical composition, as described above. In the case of treating a disease or condition, the conjugate is administered to a subject in need thereof (i.e., having or at elevated risk of having such disease or condition) in a pharmaceutically effective amount. In some embodiments, the conjugate is administered specifically to one or more cells, tissues, or organs of the subject, wherein the administration may be in vitro or in vivo. In some embodiments, the conjugate may include a targeting agent in order to target specific tissue, such as a specific organ. Targeting agents are well known in the art, and such targeting agents can be incorporated into the conjugates described herein by means well known in the art.

In further aspects, the present disclosure is directed to methods for treating or preventing a disease, disorder, or condition in a subject by administering to the subject a pharmaceutically effective amount of a conjugate described herein. An effective amount of the conjugate, typically in a pharmaceutical composition, may be administered to a human or an animal in need thereof by any of a number of well-known methods. For example, the molecule may be administered systemically or locally, such as by injection. The systemic administration of the molecule may be by intravenous, subcutaneous, intraperitoneal, intramuscular, intrathecal, or oral administration. In some embodiments, the subject exhibits one or more signs or symptoms associated with a genetic neuropathy, a genetic based musculopathy, a genetic eye disease or disorder, a genetic lung disease or disorder, a genetic liver disease or disorder, neurodegenerative disease, or cancer.

In some embodiments, the subject exhibits one or more signs or symptoms associated with Angelman syndrome, HIST1H1E (H₁-4) syndrome, Prader-Willi syndrome, Alzheimer's disease, Huntington's disease, Parkinson's Disease (PD), Multiple Sclerosis (MS), Cerebral Palsy (CP), Spinocerebellar Ataxias, Pick's disease, Gerstmann-Straussler-Scheinker disease, Hallervorden-Spatz disease, Jakob-Creutzfeldt disease, dystonia, amyotrophic lateral sclerosis (ALS), muscular atrophies, muscular dystrophies (such as Duchenne, Becker, facioscapulohumeral, myotonic, congenital, distal, Emery-Dreifuss, oculopharyngeal, and limb girdle), congenital myopathies (such as central core, Myotubular, Nemaline, Ullrich/Bethlem, and RyR1), metabolic disorders (such as Pompe's disease), Retinitis pigmentosa, choroideremia, Stargardt disease, cone-rod dystrophy, Leber congenital amaurosis, cystic fibrosis, primary ciliary dyskinesia, alpha-1-antitrypsin deficiency, surfactant metabolism dysfunction 1-4, familial pulmonary fibrosis, pulmonary alveolar microlithiasis, Dyskeratosis congenita, neurofibromatosis type I, tuberous sclerosis/LAM, Birt-Hogg-Dubé Syndrome, Hyper IgE syndrome, Hermansky-Pudlak syndrome, Gaucher disease type I, Niemann-Pick disease type B, and Lysinuric protein intolerance, hemochromatosis, Wilsons disease, alpha-1-antitrypsin deficiency, or cancer.

In more particular embodiments, the subject exhibits one or more signs or symptoms associated with Angelman syndrome, HIST1H1E (H₁-4) syndrome, Prader-Willi syndrome, Alzheimer's disease, Huntington's disease, Parkinson's Disease (PD), Multiple Sclerosis (MS), Cerebral Palsy (CP), Spinocerebellar Ataxias, Pick's disease, Gerstmann-Straussler-Scheinker disease, Hallervorden-Spatz disease, or Jakob-Creutzfeldt disease, or more particularly, the subject exhibits one or more signs or symptoms associated with Angelman syndrome, HIST1H1E (H₁-4) syndrome, or Prader-Willi syndrome.

In particular embodiments, the subject has a neurodegenerative disease. The neurodegenerative disease may be any of those mentioned above, e.g., Alzheimer's disease (including presymptomatic form, based on presence of markers), Huntington's disease, Parkinson's Disease (PD), Multiple Sclerosis (MS), ALS, dementia (including early onset, mild, or late stage, and Lewy body dementia), mild cognitive impairment, or a condition that places the subject at high risk of neurodegenerative disease (e.g., traumatic brain injury).

In other particular embodiments, the subject has a form of cancer. The cancer can be characterized by the type of cancerous tissue. The cancerous tissue may be located in any part of the body, such as, for example, the prostate, breast (including triple negative breast cancer), brain, lungs, stomach, intestines, colon, rectum, ovaries, cervix, pancreas, kidney, liver, skin, lymphs, bones, bladder, or uterus. The cancer can also include the presence of one or more carcinomas, sarcomas, lymphomas, blastomas, or teratomas (germ cell tumors).

An effective amount of a pharmaceutical composition of the invention is any amount that is effective to achieve its purpose, typically to halt or slow down the progression of a disease or condition. In some embodiments, the pharmaceutical composition at least partially reverses the deleterious effects of a disease or condition, such as by reducing the size of a tumor or reducing the level of biomarkers associated with a neurodegenerative disease. The effective amount of the conjugate, typically expressed in mg/kg, can be determined by routine methods during pre-clinical and clinical trials by those skilled in the art. Dosing is dependent on the severity and responsiveness of the infection being treated, with the course of treatment lasting from several days to several months, or until a cure or a diminution of the disease state is achieved. Optimal dosing schedules can be calculated from measurements of drug accumulation in the body of the patient. The administering physician can determine optimum dosages, dosing methodologies, and repetition rates. In different embodiments, depending on these and other factors, a suitable dosage of the active ingredient may be precisely, at least, or no more than, for example, 1 mg, 10 mg, 50 mg, 100 mg, 200 mg, 300 mg, 400 mg, 500 mg, 600 mg, 700 mg, 800 mg, 900 mg, 1000 mg, 1200 mg, or 1500 mg, per 50 kg, 60 kg, or 70 kg adult, or a dosage within a range bounded by any of the foregoing exemplary dosages. Depending on these and other factors, the composition is administered in the indicated dosage by any suitable schedule, e.g., once, twice, or three times a day (or once every two or three days or per week) for a total treatment time of one, two, three, four, or five days, and up to, for example, one, two, three, or four weeks, or 1, 2, 3, 4, 5, 6, or 12 months. The indicated dosage may alternatively be administered once every two or three days, or per week. Alternatively, or in addition, the composition is administered until a desired change is evidenced.

Examples have been set forth below for the purpose of illustration and to describe certain specific embodiments of the invention. However, the scope of this invention is not to be in any way limited by the examples set forth herein.

### EXAMPLES

### Overview

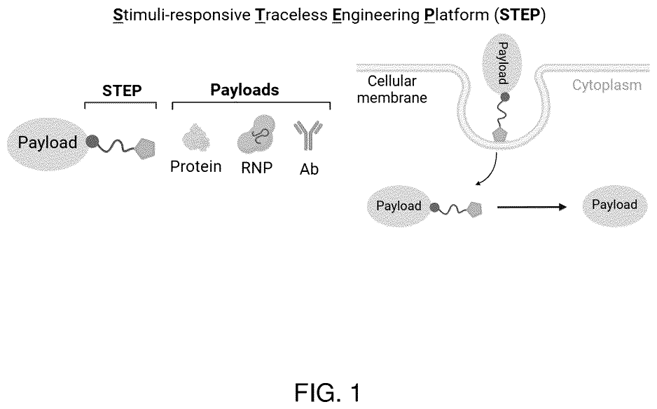

The present disclosure reports a STEP technology for the delivery of payloads into cells. As shown in FIG. 1, payloads are conjugated to STEP molecules to form the payload-STEP, which can effectively enter cells to release the payload for biological function.

The experimental results and figures are organized based on the sequence of each STEP component in the final claims: (1) Aromatic and nonaromatic binding domains R1 screening in reporter cells, the noncovalent binding mechanism is also included; (2) Aromatic and fatty acidic cell penetration domains R3 screening in Ai9 reporter cells; (3) Traceless linker —S—S—, branched STEP, linker length, R2a-R2b conjugation chemistry, “1 step” vs. “2 step” formulation chemistry; (4) Delivered payloads, interactions with cells and physiochemical characterization.

### Example 1. Aromatic Binding Domains for the STEP Delivery Platform

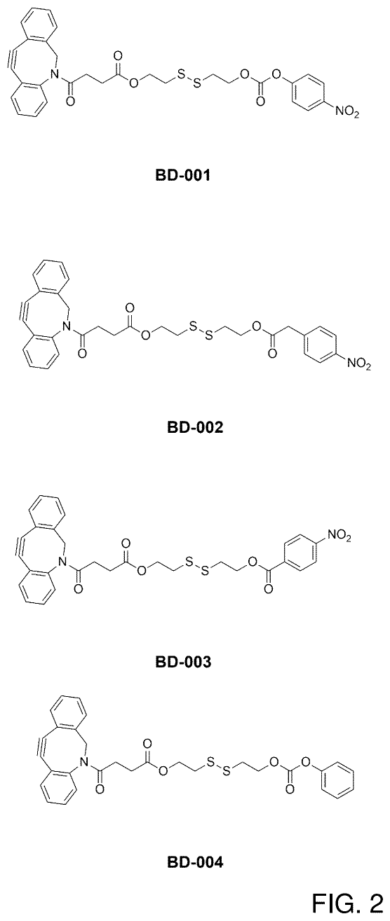

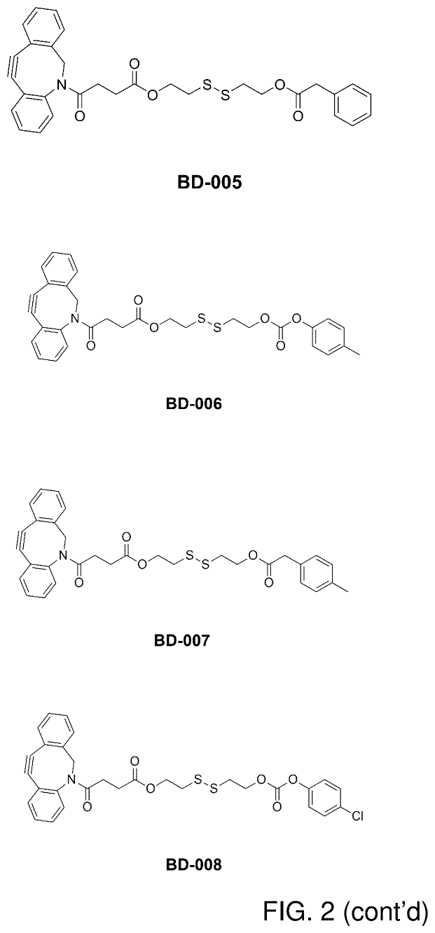

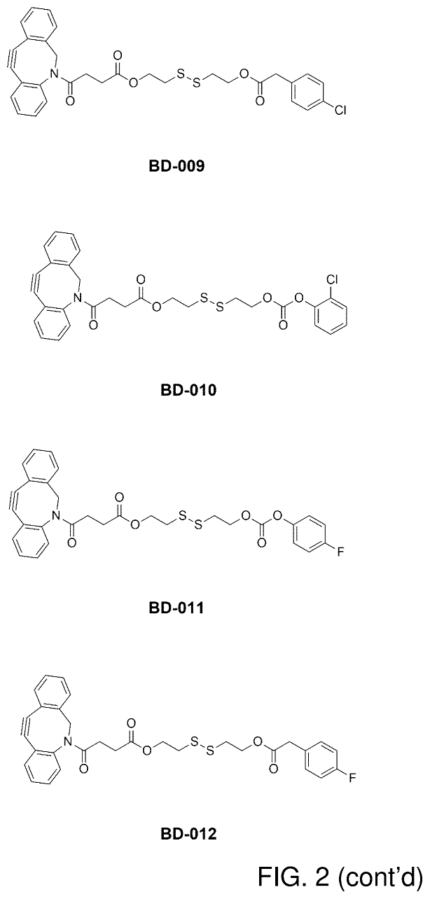

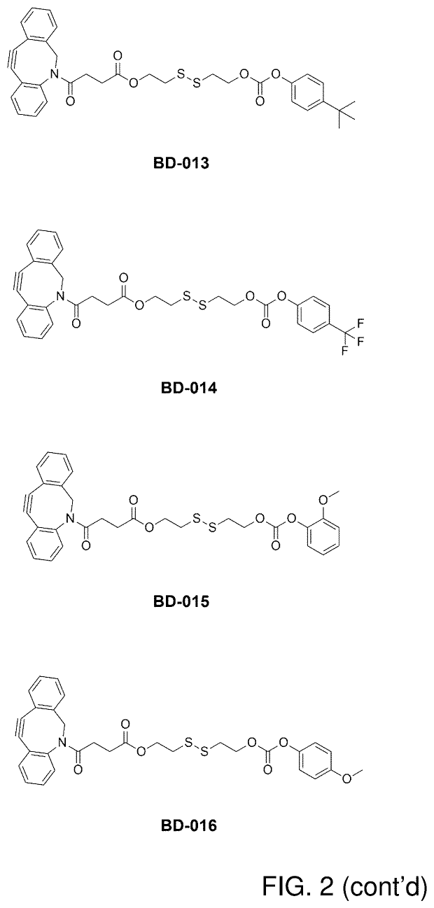

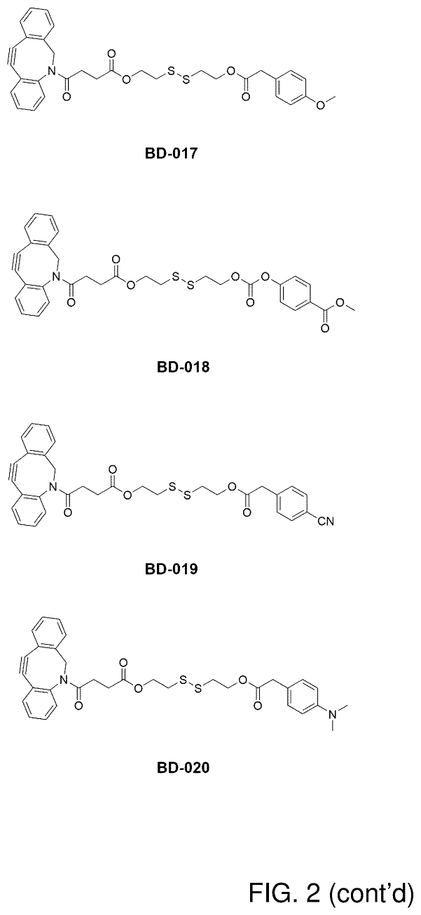

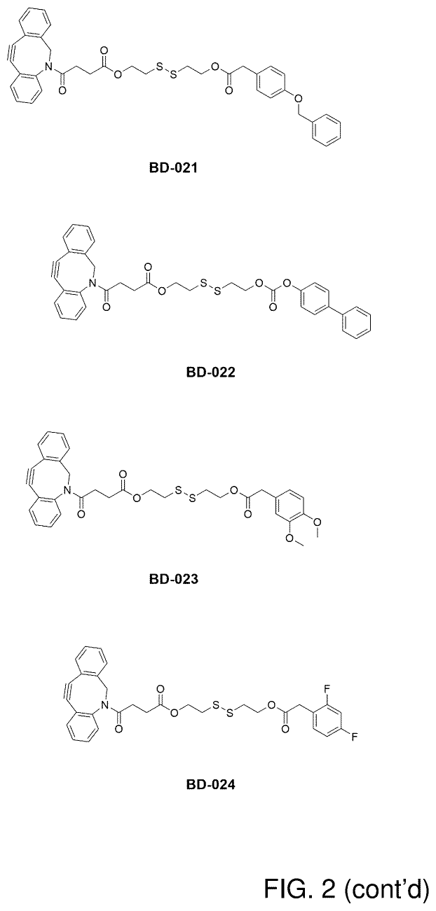

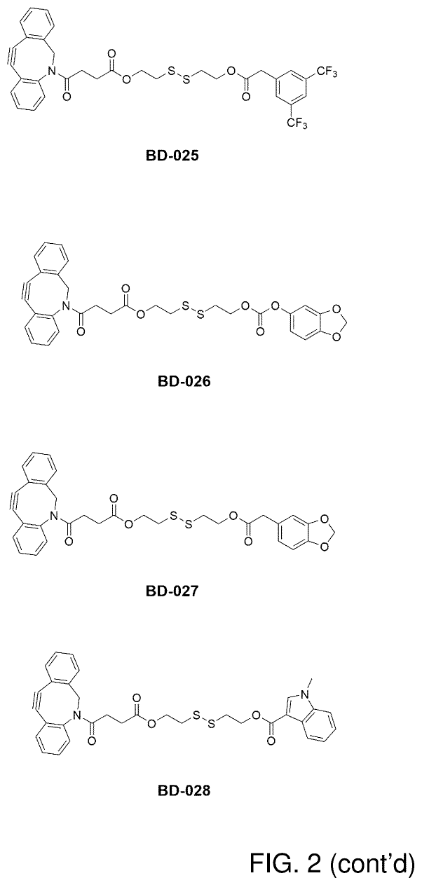

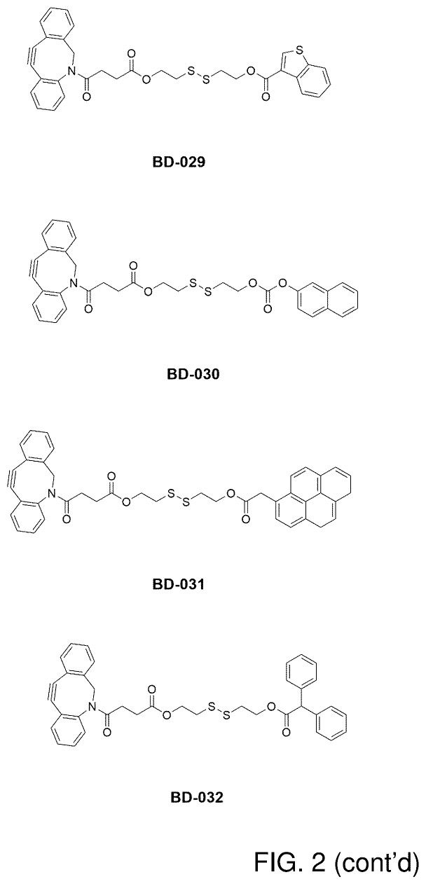

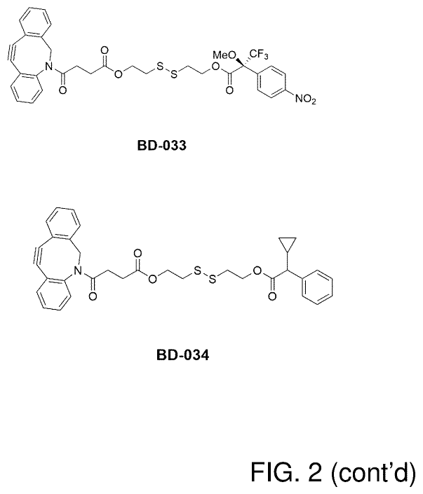

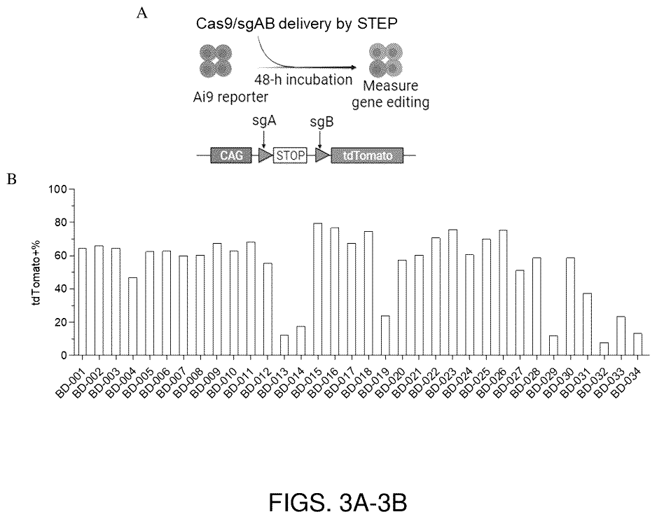

STEP molecules are designed to conjugate with the payloads through chemical linkers or binding domains. As shown in FIG. 2, a library of STEP molecules with various ending aromatic chemical linkers or binding domains R1 were synthesized. For example, Compound BD-001 with DBCO-nitrophenyl carbonate (NPC) structure has the potential to react with primary amine group in payloads. However, BD-002 with DBCO-4-Nitrophenyl acetate (NPA) structure and BD-003 cannot react with primary amine group. The efficiency of these different STEP molecules were tested with the same cell-penetration domain to deliver RNP. As shown in FIG. 3A, Ai9 reporter cells were treated with STEP RNP carrying sgRNA targeting Ai9 (sgAB) at Cas9 concentration of 10 μg/mL. After treatment for 48 h, the effectively edited Ai9 reporter showed the fluorescence of tdTomato expression. FIG. 3B showed the delivery efficiency of these STEP molecules.

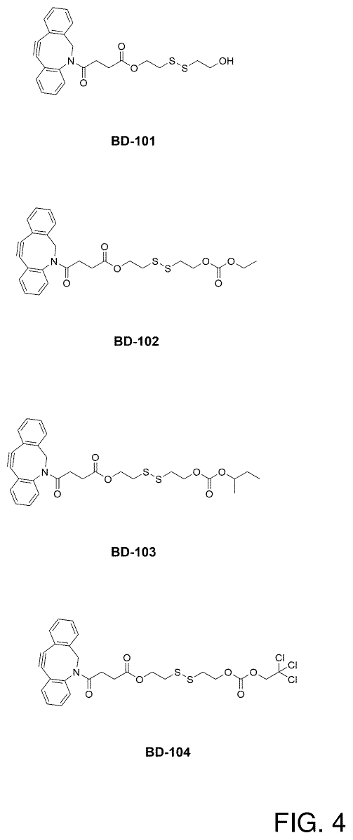

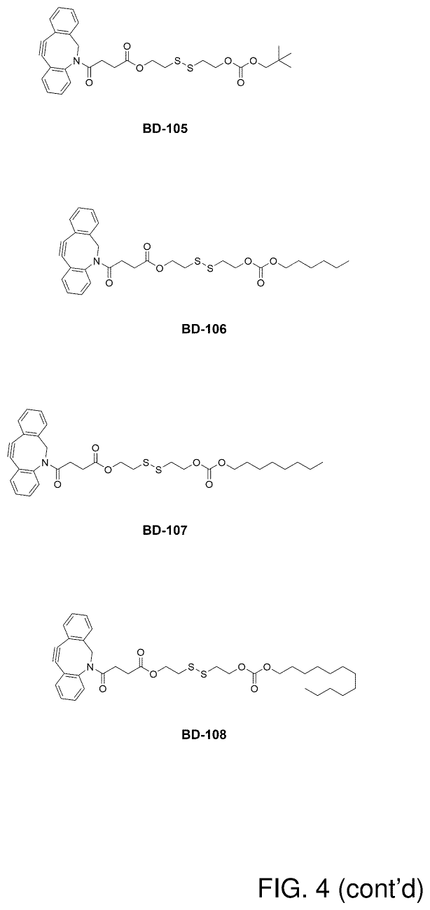

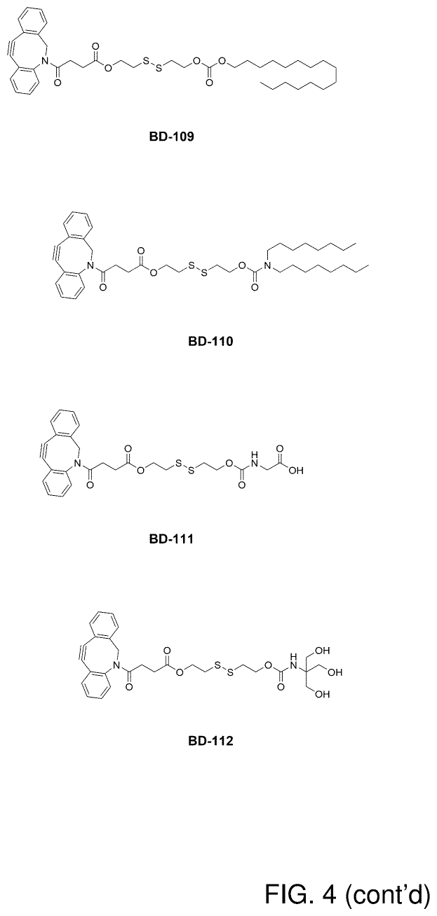

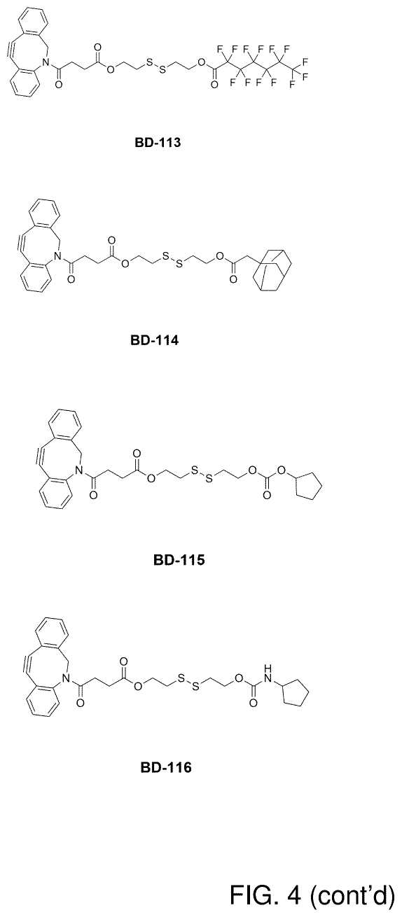

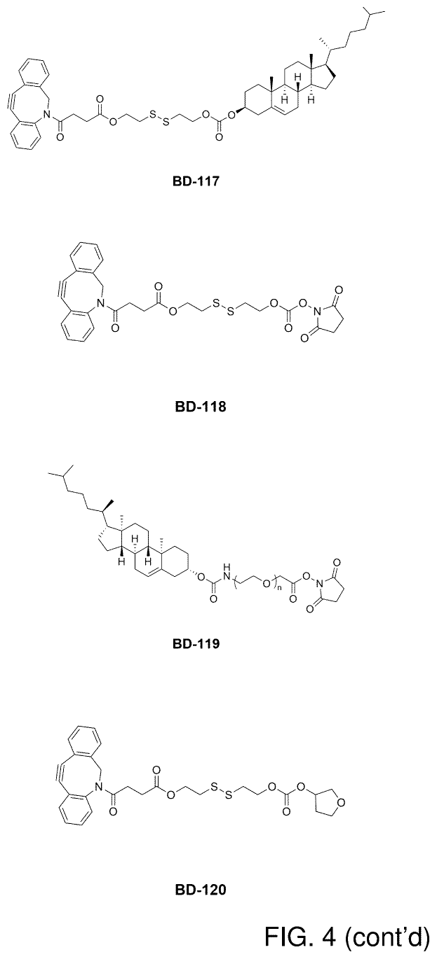

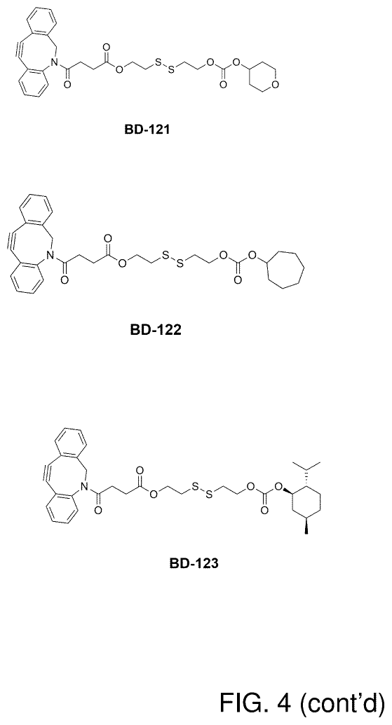

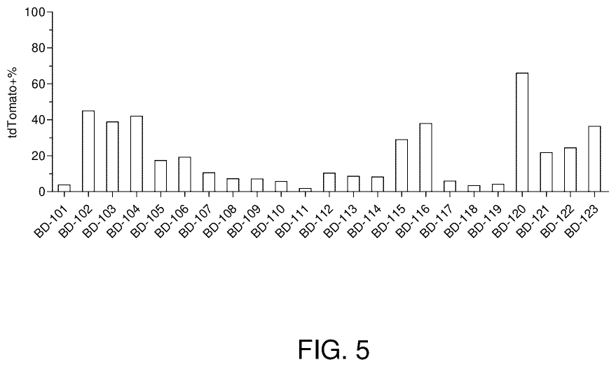

As a control, STEP with no aromatic ending group (BD-101) showed almost no editing efficiency (FIGS. 4-5). STEP molecules with an aromatic ending group, such as BD-001, normally showed effective ending efficiency in the reporter cells with over 60% efficiency. These results confirm that the aromatic group chemistry structure greatly contributes to the delivery efficiency.

Suitable substituent group on benzene ring can increase the editing efficiency. For example, BD-015, BD-016, BD-018, BD-023, and BD-026 showed higher editing efficiency than other tested molecules. These optimized STEP molecules outperform other STEP molecules for gene delivery.

Large aromatic chemical ending groups can reduce the editing efficiency. For example, BD-031 with pyrene structure decreased the editing efficiency in comparison other single aromatic structure. These results have high value for further optimization of STEP molecules.

### Example 2. Nonaromatic Binding Domain for STEP Delivery Platform

As shown in FIG. 4, a library of STEP molecules with various ending non-aromatic chemical linkers or binding domains R1 were synthesized. The efficiency of these different STEP molecules were tested with the same cell-penetration domain to deliver RNP in Ai9 reporter cells. FIG. 5 shows the delivery efficiency of these STEP molecules. The following deductions can be made: (1) STEP with nonaromatic R1 groups can effectively deliver RNP. For example, BD-120 can edit over 60% cells. (2) R1 with longer carbon chain length has lower efficiency in comparison with R1 with short carbon chain length. For example, BD-102 has higher editing efficiency than BD-109. (3) Heterocyclic groups normally have lower delivery efficiency than benzyl group. BD102, BD-103, BD-104, BD-116, BD-120 and BD-123 are the optimized nonaromatic R1 for STEP mediated gene delivery.

### Example 3. STEP Binds to the Delivered Cargo by a Non-Covalent Interaction

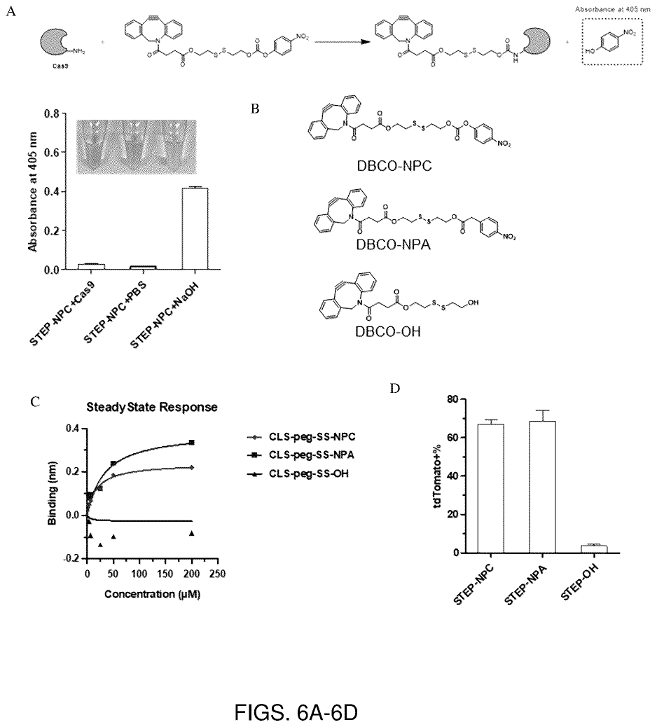

Although the selected STEP molecule contains NPC, an amine-reactive group, it interacts with RNPs through a non-covalent mechanism. Incubation of the STEP molecule with RNPs did not result in the release of free NPCs, which would have been detectable if a nucleophilic attack had occurred during a covalent reaction (FIG. 6A). Biolayer interferometry (BLI) analysis revealed that the NPC-terminated STEP interacts with the Cas9 protein, with an equilibrium dissociation constant (K_D) of 17.8 μM. A STEP molecule terminated with 4-Nitrophenyl acetate (NPA), which has minimal structural differences from NPC but lacks amine reactivity, demonstrated a similar binding affinity and comparable ability to deliver RNPs (FIGS. 6B-6C). Maintenance of the nitrophenyl structure is crucial for the binding activity of STEP molecules, as the hydrolyzed form of NPC STEP lacking the aromatic ring did not exhibit detectable binding under the same testing conditions (FIGS. 6C-6D).

### Example 4. Aromatic Cell Membrane Penetration Domain for STEP Delivery Platform

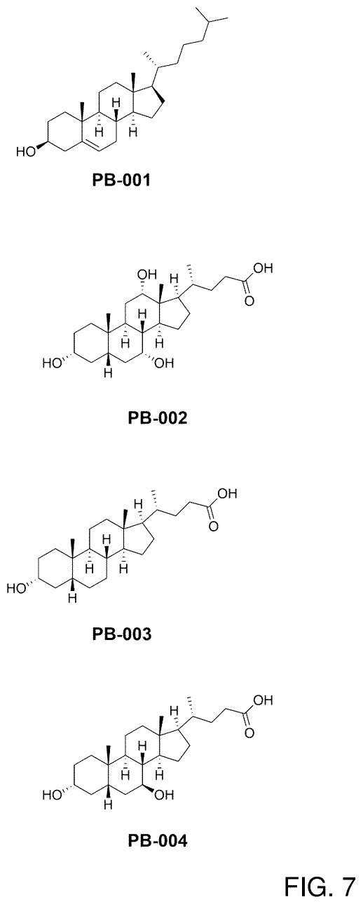

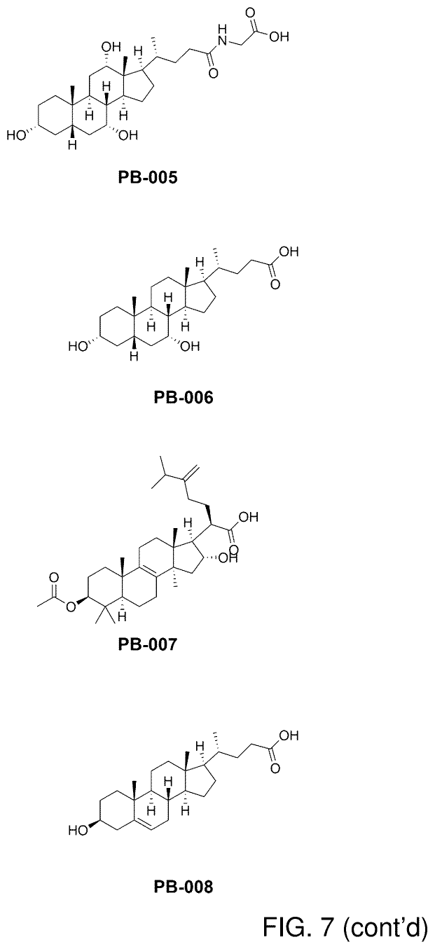

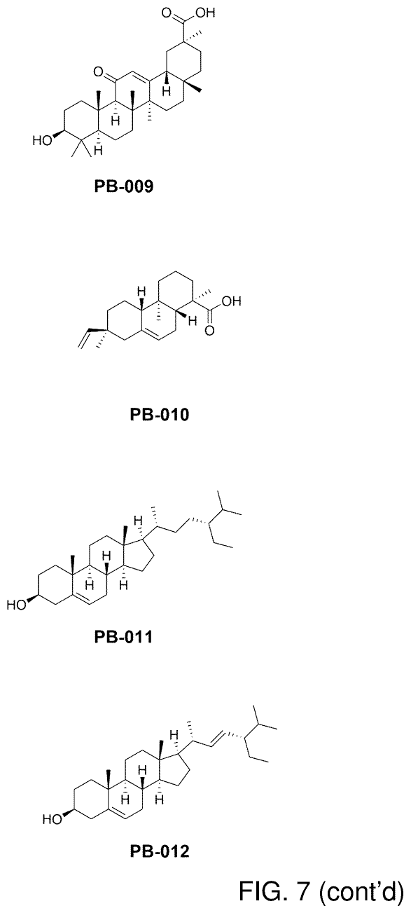

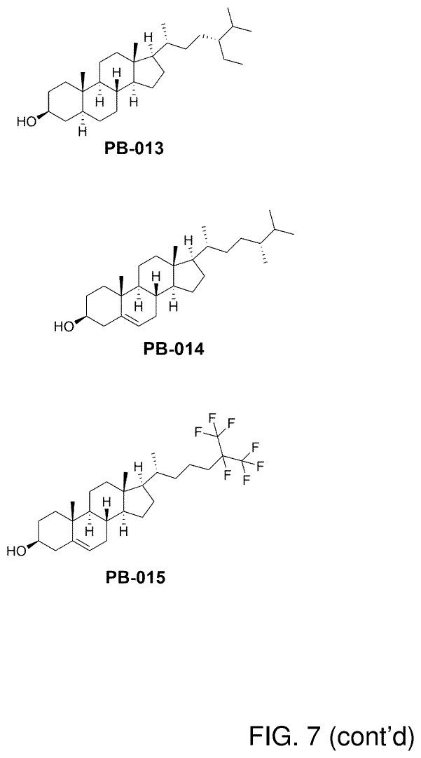

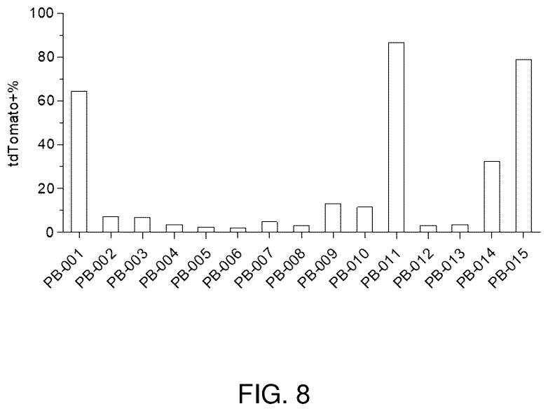

STEP molecules are designed to deliver the payloads into cells through the cell penetration domain R3. FIG. 7 shows a library of STEP molecules with various cell membrane penetration domains R3 that were synthesized. These R3 molecules mainly include cholesterol and its chemical analogs. The efficiency of these different STEP molecules were tested with the same binding domain R1 to deliver RNP in Ai9 reporter cells. FIG. 8 shows the delivery efficiency of these STEP molecules. Cholesterol STEP showed highly effective gene editing in Ai9 reporter cells. Among the tested R3 molecules, β-sitosterol and F7-Cholesterol showed better delivery efficiency than cholesterol. Cholesterol, β-sitosterol and F7-Cholesterol are the optimized aromatic cell membrane penetration domain for STEP gene delivery.

### Example 5. Fatty Acidic Cell Membrane Penetration Domain for STEP Delivery Platform

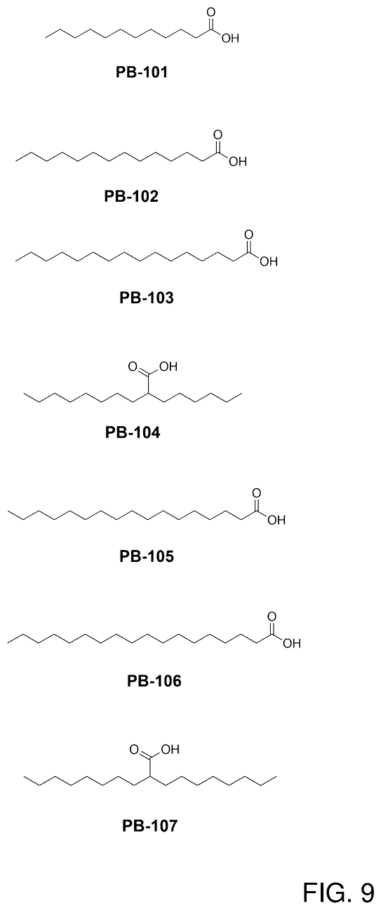

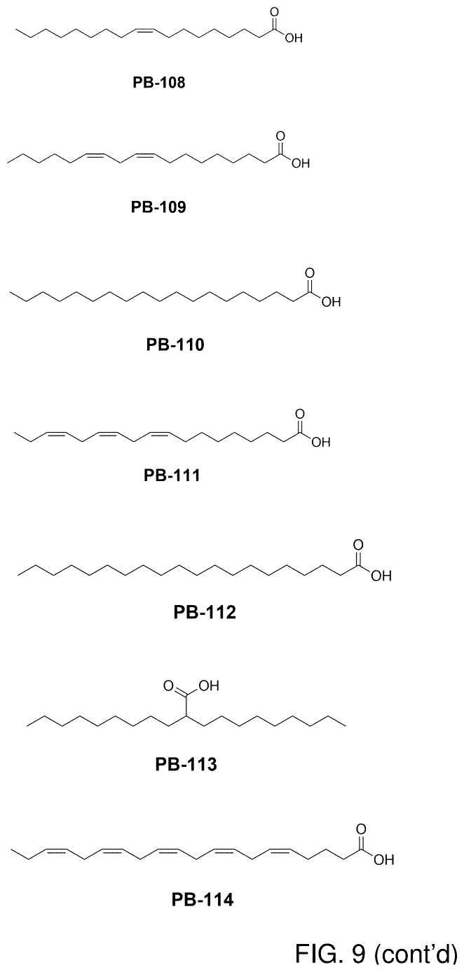

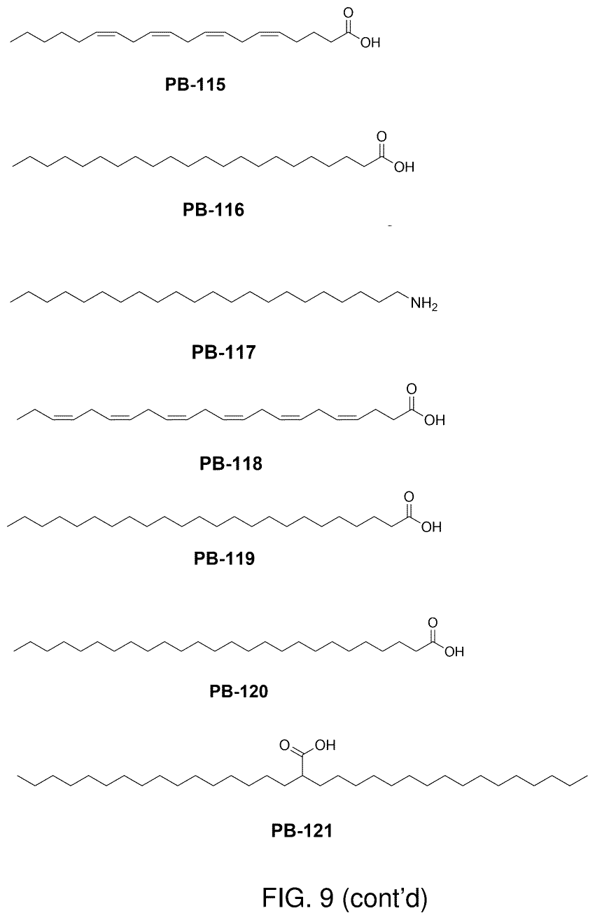

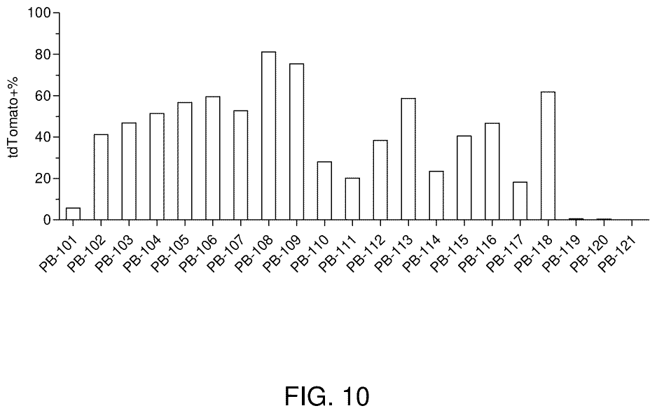

As shown in FIG. 9, a library of STEP molecules with various fatty acidic cell membrane penetration domain R3 were synthesized. The efficiency of these different STEP molecules were tested with the same binding domain to deliver RNP in Ai9 reporter cells. FIG. 10 showed the delivery efficiency of these STEP molecules. The following deductions can be made: (1) Fatty acid carbon chain length affects the delivery efficiency. Carbon length at C13-C22 showed effective gene editing in Ai9 reporter cells. (2) A few double carbon bonds can increase the delivery efficiency. For example, PB-108 (C17, one double bond) and PB109 (C17, two double bonds) showed higher editing efficiency than PB-107 (C17, zero double bond). (3) C17 fatty acid with one or two double bonds PB-108 and PB-109 are the optimal fatty acidic cell membrane penetration domains for STEP delivery platform.

### Example 6. Traceless Linker for Increased STEP Delivery Efficacy

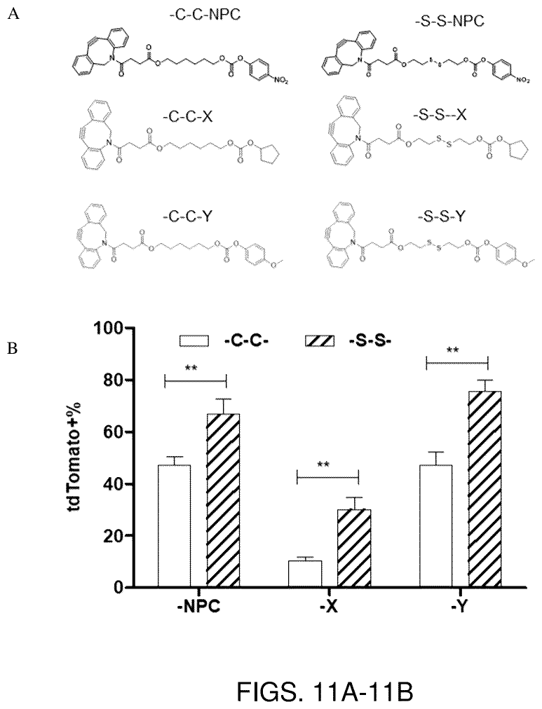

FIGS. 11A and 11B show the effect of traceless linker on the delivery efficiency. Incorporating a disulfide bond within the protein-binding domain enhances delivery efficiency. FIG. 11A shows the chemical structure of the traceless linkers (—S—S—) and paired untraceless linkers (—C—C—) with 3 different binding molecules including -NPC, -X, and -Y, As shown by the gene editing delivery efficiency results in FIG. 11B, compared to those without a disulfide bond, STEP molecules with a disulfide bond showed superior delivery efficiency. This enhancement is likely due to the cleavage of the disulfide bond by glutathione (GSH), which is abundant in the cytoplasm, allowing for traceless release of the RNP payload.

### Example 7. Branched STEP for Effective Gene Delivery

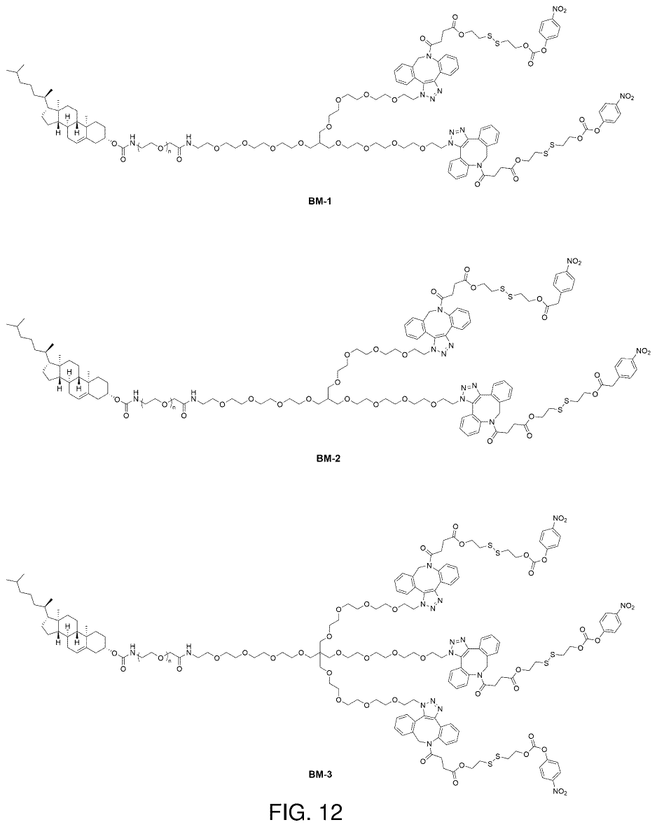

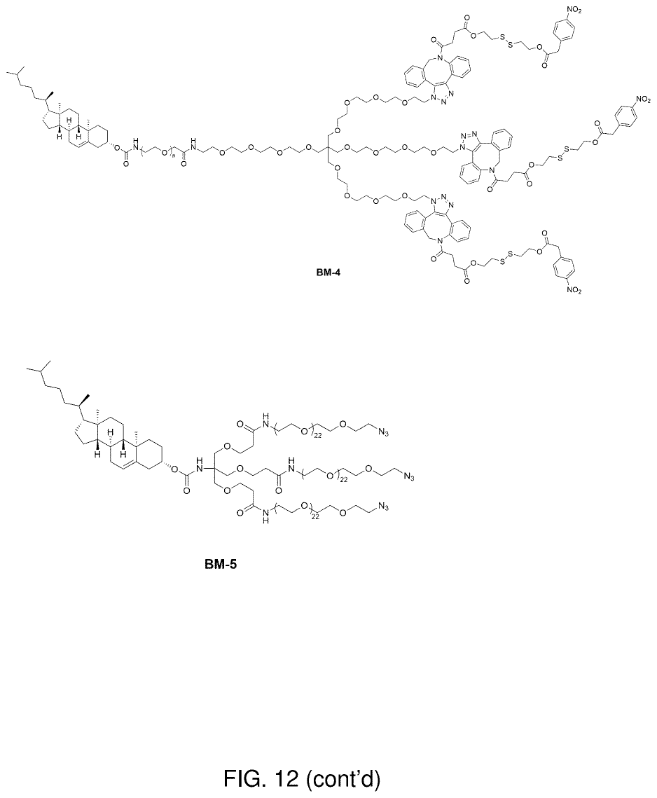

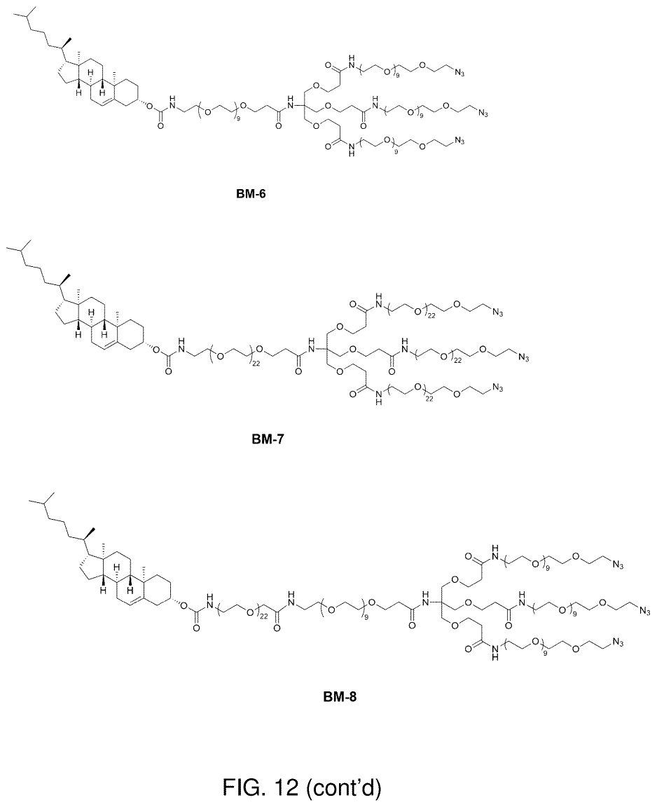

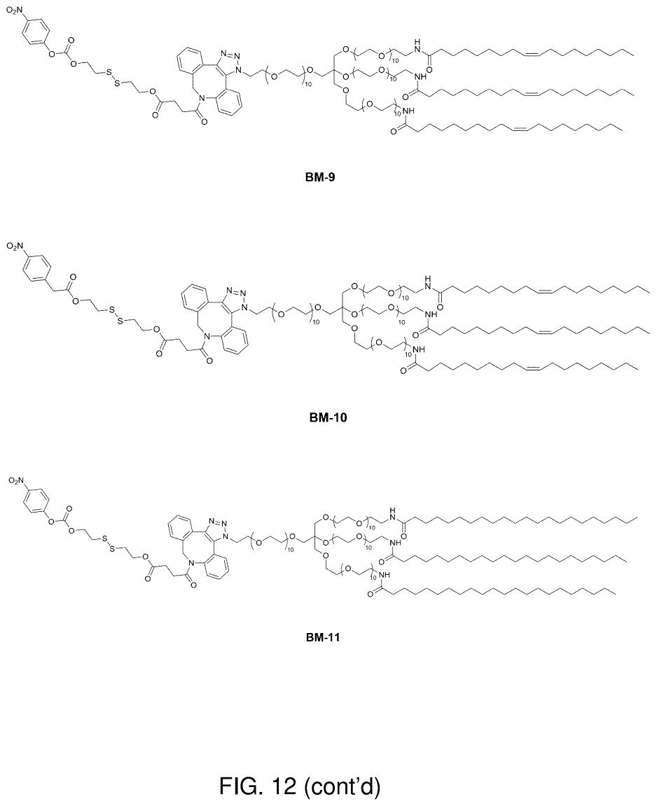

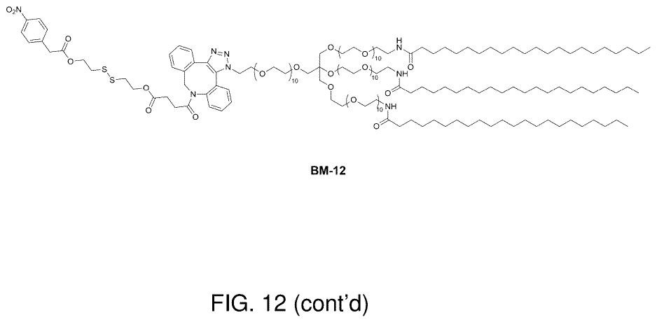

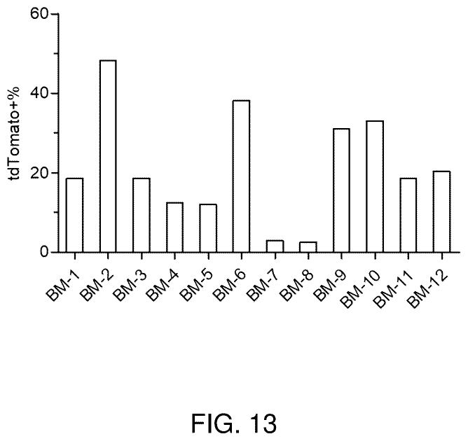

Branched structures possibly increase both the binding efficiency of STEP to payloads and the penetration activity to cells. FIG. 12 shows the molecular structures of different branched STEP. FIG. 13 shows the delivery efficiency of these branched STEP. BM-2, BM-6, BM-9, BM-10 are the optimal branched STEP for STEP delivery platform.

### Example 8. The Effect of Linker Length on STEP Delivery Platform

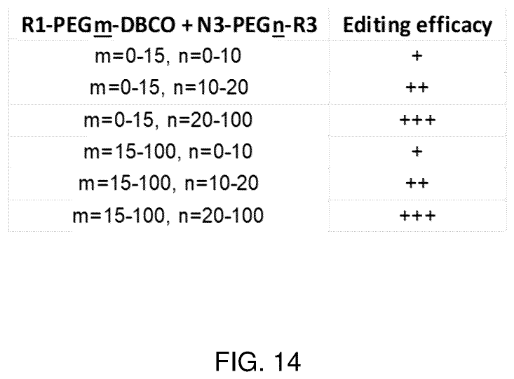

FIG. 14 shows the effect of linker length affects the delivery efficiency. Length of the binding domain and binding domain is critical. DBCO-PEGm-R1 prefers PEGO-PEG15 between DBCO and R1 for effective cell editing. R3-CLS-PEGn with PEGn=20-100 shows the optimized editing activity.

### Example 9. The Effect of Chemical Conjugation R2a-R2b Between R1 and R3 on STEP Delivery Platform

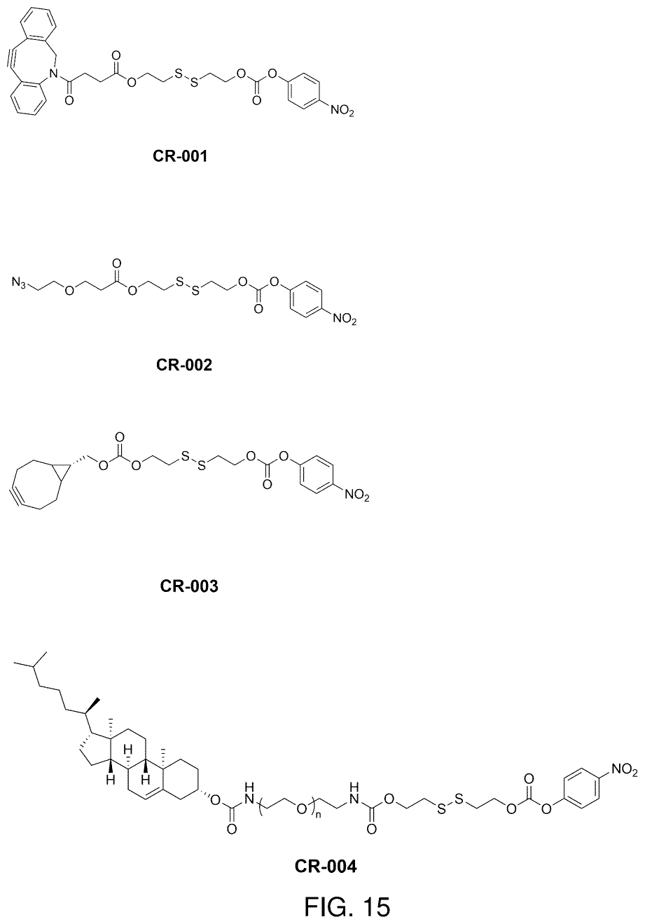

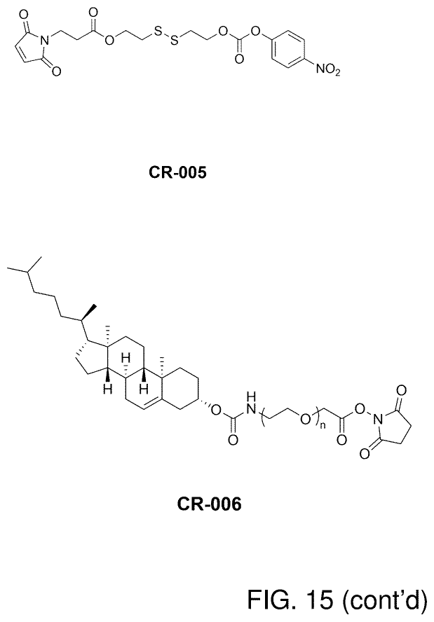

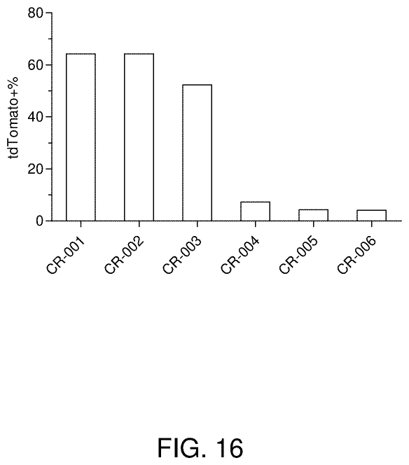

The chemical conjugation R2a-R2b between binding domain R1 and cell penetration domain R3 also affects the delivery efficacy. FIG. 15 shows various conjugation chemistries, including DBCO-N₃ linker, N₃-DBCO linker, non-DBCO click chemicals and non-click chemicals. FIG. 16 shows the delivery efficacy of the STEP with the tested conjugation chemistry. Click conjugation outperformed other conjugation chemicals. DBCO-azide chemistry showed better results than other click chemicals. The DBCO-azide location in STEP molecules did not affect the delivery efficiency. These results confirm that DBCO click chemicals show higher efficiency than non-DBCO click chemicals and non-click chemicals.

### Example 10. The Effect of Formulation Chemistry on STEP Delivery Platform Efficacy

The conjugation of STEP molecules to payload can be achieved through two different processing methods. “1 step” (or “one-step”) conjugation proceeds by the single step assembly of the complete STEP molecule with the payload. “2 step” (or “two-step”) conjugation includes the following two steps: the first step is the binding of the binding domain R1 with payload; the second step is the chemical conjugation of penetrating domain to the binding domain.

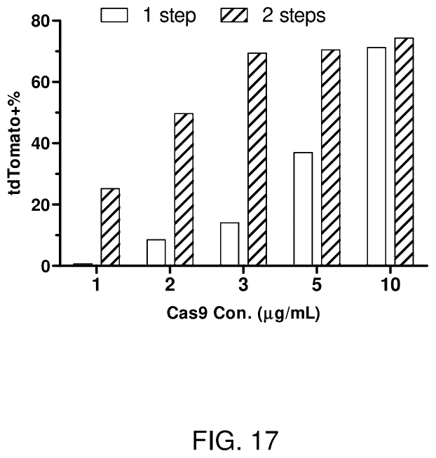

The comparison results of the two conjugation methods in delivery efficiency are shown in FIG. 17. Both conjugation methods STEP RNP can achieve similarly high editing efficiency over 70% at high Cas9 concentration of 10 μg/mL. But at a lower Cas9 concentrations, the “2 steps” method showed much higher editing efficiency than the standard “1 step” method. For example, the “2 steps” method showed 70% editing efficiency at 3 μg/mL. But the “1 step” method showed only 14% editing efficiency at 3 μg/mL. The results confirmed the “2 steps” conjugation STEP method has a higher potency than the “1 step” method STEP.

### Example 11. Delivery of Various Molecular Weight Cargos by STEP Delivery Platform

STEP technology has been demonstrated to deliver cargos with various molecular weights, including small Mw protein Cre (38.5 kDa), large Mw antibody (150 kDa) and dCas9 (260 kDa).

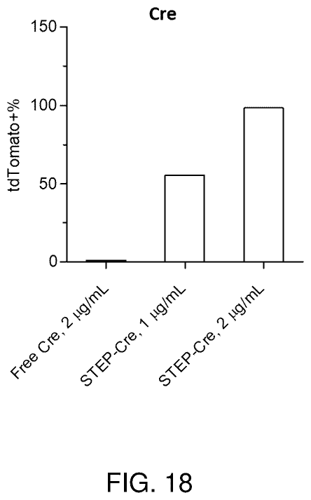

FIG. 18 shows the delivery of Cre by STEP in Ai9 reporter cells. The data show that STEP-Cre can effectively edit over 98% Ai9 reporter cells at 2 μg/mL, while free Cre did not edit Ai9 cells at 2 μg/mL.

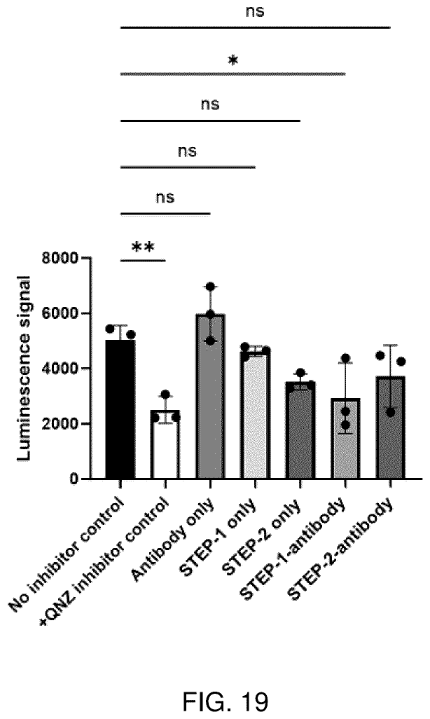

FIG. 19 shows that STEP is capable of delivering antibody into cells based on the 4*NF-κB-luciferase reporter assay. IgG antibody was conjugated to STEP and added to NF-κB-RE-luc2P HEK293 cells. TNF-α were added to induce reporter gene expression. After 5 hours of induction in a tissue culture incubator, luciferase activity was quantified using the Luciferase Assay System Reagent. The results showed that STEP-1, which is composed of β-sitosterol and NPC, was able to effectively deliver the antibody into cells to target cellular NF-κB signaling.

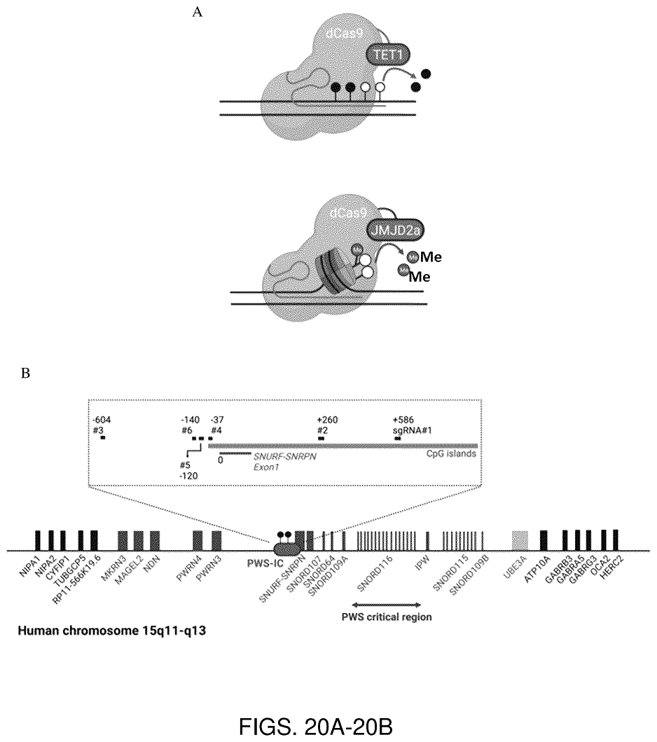

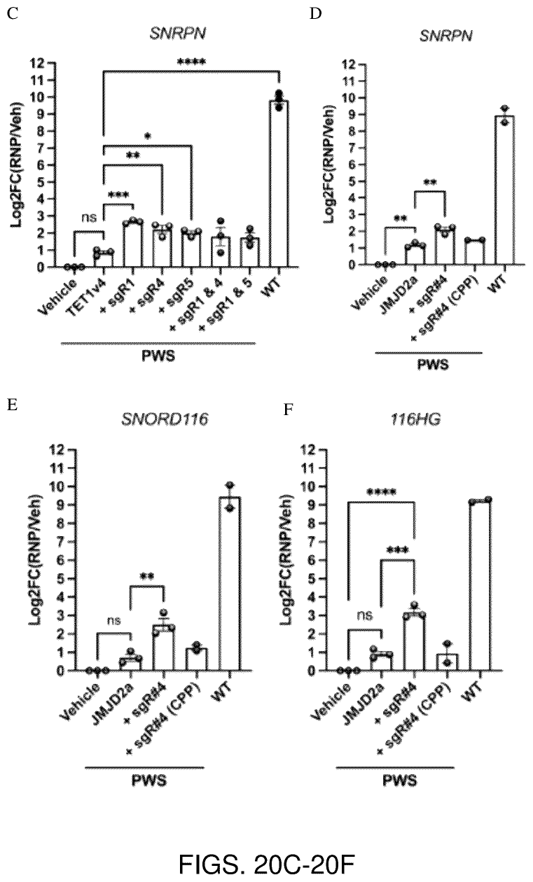

FIGS. 20A-20F show the effective delivery of dCas9 by STEP in cells. As shown in FIGS. 20A and 20B, dCas9-TET1 was treated with sgRNA packaged by STEP to fibroblasts derived from patient with Prader-Willi Syndrome which has a large deletion on paternal chromosome 15q11-q13. As shown in FIG. 20C, this ribonucleoprotein reactivated maternal imprinted/silenced gene, SNPRN. As shown in FIGS. 20D, 20E, and 20F, dCas9-JMJD2a with sgRNA packaged by STEP showed more efficacy on reactivation of imprinted genes in PWS fibroblasts compared to CPP (cell penetration peptide). dCas9-JMJD2a with sgRNA could reactivate SNRPN, SNORD116, 116HG (host gene) from the maternal chromosome.

### Example 12. The Internalization Mechanism of STEP Delivery Platform into Cells

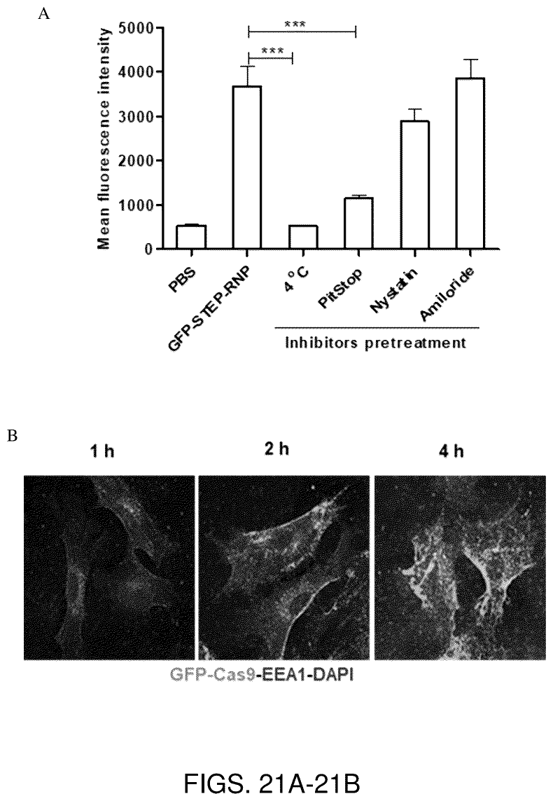

The internalization route of STEP delivery platform into cells were studied by pretreatment with various inhibitors. FIG. 21A shows results of quantitative flow cytometric analysis of cells after incubation with STEP GFP-Cas9/sgNC RNP. The results confirm that STEP-RNPs penetrated cells mainly through clathrin-dependent endocytosis pathway. The confocal microscopy study result in FIG. 21B further demonstrates the following of endocytosis with rapid and efficient endosomal escape and nuclear transport of the payload Cas9 RNP.

### Example 13. The Interaction of STEP Delivery Platform with Cells

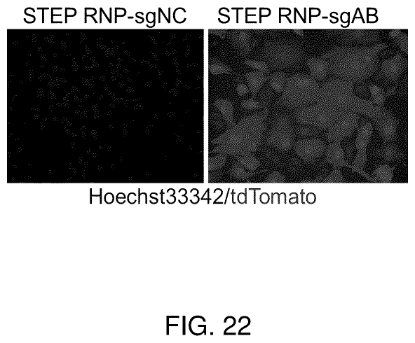

Ai9 reporter cells were treated with STEP RNP carrying sgRNA targeting Ai9 (sgAB) or scramble sgRNA (sgNC) at Cas9 concentration of 15 μg/mL. FIG. 22 shows a fluorescent image of tdTomato expression in Ai9 reporter cells after treatment. The STEP RNP-sgNC did not edit any cells. STEP RNP-sgAB achieved up to 80.3% editing in Ai9 reporter cells. This result confirms that the interaction of STEP delivery platform with cells is capable of delivering cargos into cells for its biological activity.

### Example 14. Size, Surface Charge, and Morphology of STEP-RNP

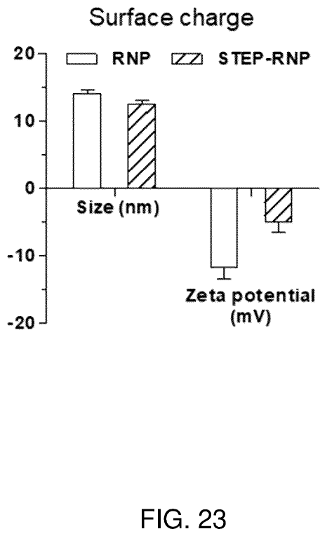

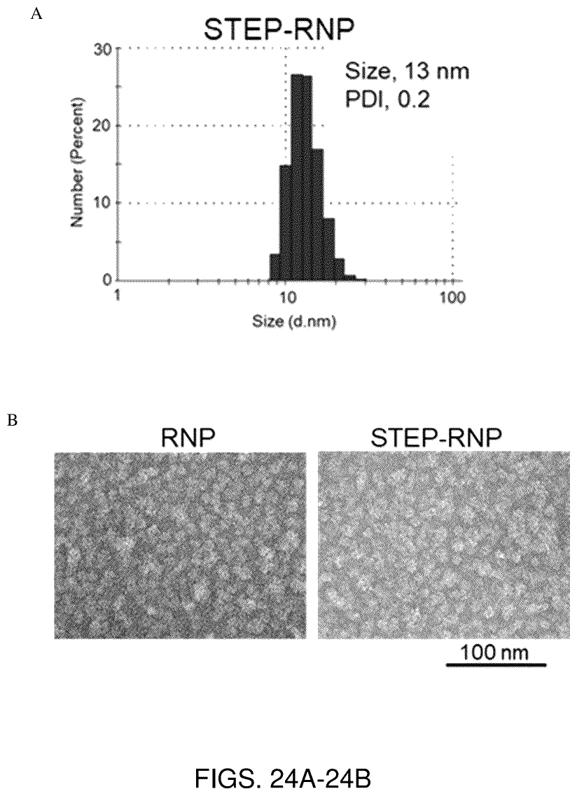

As shown by the dynamic light scattering (DLS) measurements in FIG. 23, the size of Cas9/sgRNA RNP was around 13 nm. The complexing with STEP did not increase the size of STEP-RNP. The surface charge of Cas9/sgRNA RNP was around-12 mV due to the excess ratio of negatively charged sgRNA to positively charged Cas9. Complexing with STEP effectively reduced the surface charge to around-5 mV. As shown in FIG. 24, STEP-RNP showed similar spherical morphology with RNP based on the transmission electronic microscopy observation.

### Example 15. Delivery of Base Editing and Prime Editing Cargos by STEP Delivery Platform

FIG. 25 is a schematic showing the working mechanism of the GFP-on (GER10) mouse model (C57BL/6N-Gt(ROSA)26Sor^{em3(CAG-Venus*)Rkuhn}/MurrMmjax; The Jackson Laboratory) as a reporter system for base editing and prime editing.

The GER10 base-editing reporter contains a GFP coding sequence with a nonsense mutation introduced by a G-to-A substitution, resulting in premature termination and production of truncated GFP. Adenine base editors (ABE) or prime editors (PE) that target this mutation can revert A to G, thereby restoring the expression of full-length GFP. STEP-ABE or STEP-PE treatment enables GFP restoration, turning cells green, which can be visualized by fluorescence microscopy. This reporter allows quantitative assessment of ABE PE editing efficiency using fluorescence rather than NGS.

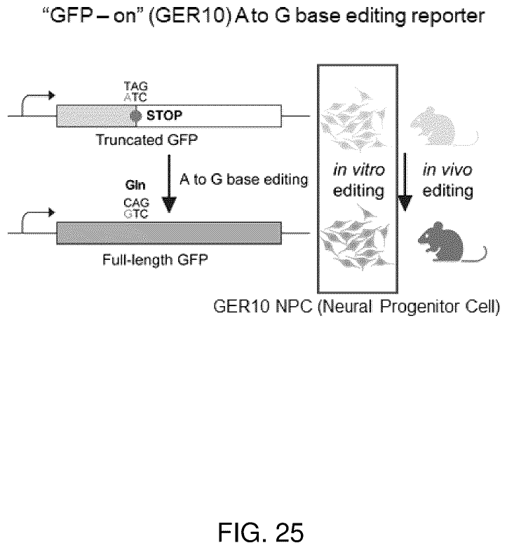

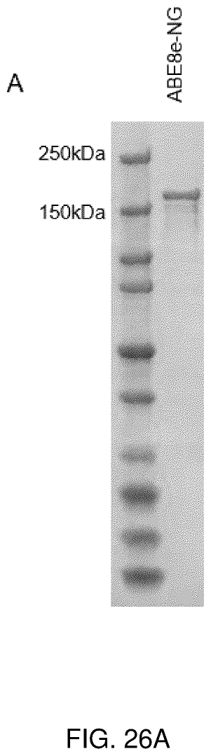

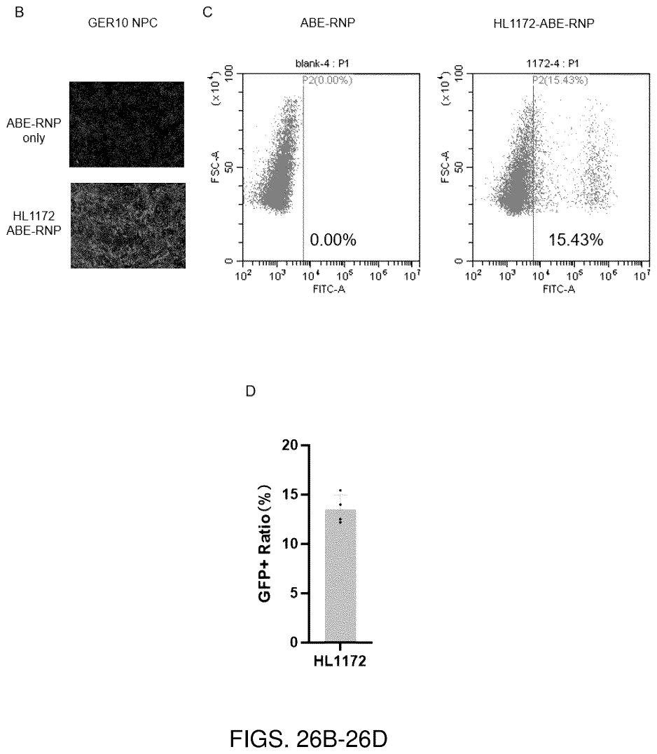

His-tagged base editor ABE8e-NG was expressed in E. coli BL21 (DE3) Star and purified using Ni-NTA resin followed by separation on a HiTrap HP column using a protein purification system. FIG. 26A shows the polyacrylamide gel electrophoresis band of ABE8e-NG protein. As shown in FIG. 26A, ABE8e-NG (~180 kDa) was robustly expressed in E. coli and efficiently purified by Ni-NTA affinity chromatography.

To assess ABE editing activity, GER10 neuronal progenitor cells (NPCs) were isolated from GER10 reporter mice and treated with STEP RNP complexes consisting of the STEP molecule H1172, ABE8e-NG, and an sgRNA targeting the A-to-G transition in the GFP reporter. Seventy-two hours after treatment, edited cells were analyzed by immunofluorescence microscopy. FIG. 26B shows the HL1172 ABE-RNP can edit GER10 NPC cells emitting green fluorescence. As shown in FIG. 26B, GFP reporter reactivation occurred only in GER10 NPCs treated with STEP (H1172)-ABE RNP, and not in cells treated with the negative-control ABE RNP lacking the STEP (H1172) molecule.

Editing efficiency was quantified by flow cytometry (FIG. 26C) using FITC gating established from untreated controls. GER10 NPCs treated with STEP (H₁₁₇₂)-ABE RNP exhibited an editing efficiency of 15.43% (FIG. 26D).

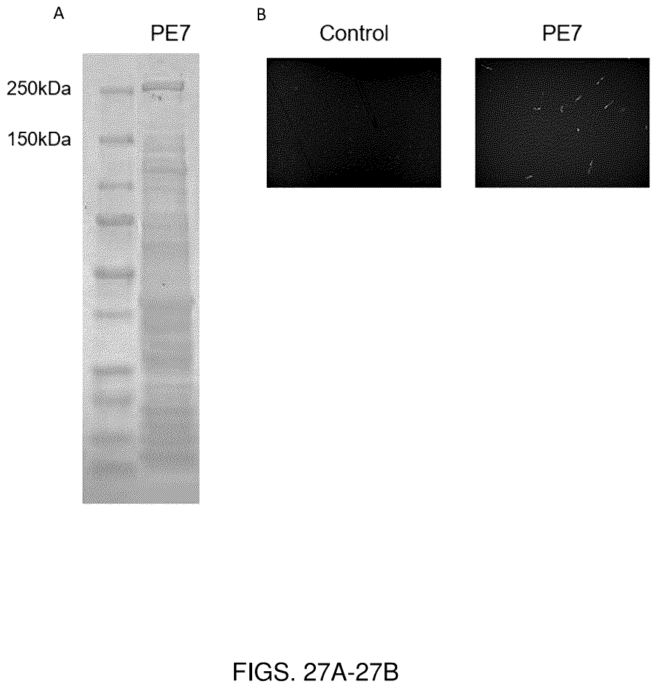

His-tagged Prime Editor 7 (PE7) was expressed in E. coli BL21 (DE3) Star and purified using Ni-NTA resin. FIG. 27A shows the polyacrylamide gel electrophoresis band of prime editor 7 (PE7) protein. FIG. 27B shows the STEP-PE-RNP edited GFP-on NPC cells emitting green fluorescence. FIG. 27A indicates that PE7 (~270 kDa) can be expressed in E. coli and purified through Ni-NTA chromatography, despite its limited purity. PE7 was assembled with epegRNAs targeting the GER10 reporter sequence and conjugated with a STEP molecule. The results show that STEP-conjugated PE RNPs can be delivered into GER 10 NPC and mediate efficient A-to-G correction (FIG. 27B).

While there have been shown and described what are at present considered the preferred embodiments of the invention, those skilled in the art may make various changes and modifications which remain within the scope of the invention defined by the appended claims.

What is claimed is:

1. A compound having the following structure: 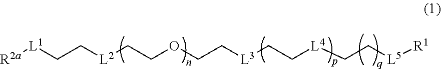
wherein:
R^{2a} is a first reactive group that forms a linkage with a second reactive group;
L¹ is a linker selected from a bond, C(O), CH₂, C(O)NH, and C(O)O;
L², L⁴, and L⁵ are linkers independently selected from a bond, CH₂, C(O), NH, O, S, C(O)NH, C(O)O, OC(O)NH, OC(O)O, NHC(O)NH, and —OC(O)O—Ar—, or a CH₂ bound to any of the foregoing linkers, wherein Ar is an aromatic ring, and wherein an O atom can optionally be replaced with an S atom in any of the foregoing linkers;
L³ is a linker selected from the group consisting of a bond, CH₂, CH₂CH₂, S—S, C(O)O, OC(O)O, C(O)NH, OC(O)O—Ar, wherein Ar is an aromatic ring, and linked combinations of any two or more of the foregoing linkers;
R¹ is an aromatic or aliphatic ring or polycyclic ring system, wherein the aromatic or aliphatic ring or polycyclic ring system optionally contains one or more ring heteroatoms selected from N, O, and S, and the aromatic or aliphatic ring or polycyclic ring system optionally contains one or more non-H substituents selected from halogen atoms; oxo; linear or branched, cyclic or acyclic, saturated or unsaturated, halogenated or non-halogenated hydrocarbon groups containing 1-30 carbon atoms; NO₂; NR³R⁴; CN; C(O)R⁵; C(O)OR⁶; OC(O)R⁶; OR⁷; C(O)NR³R⁴; and NHC(O)R⁸;
wherein R³, R⁴, R⁵, R⁶, R⁷, and R⁸ are independently selected from H and linear or branched alkyl groups containing 1-3 carbon atoms;
and R¹ is optionally connected to L⁵ by a CR^{a}R^{b} linker, wherein R^{a} and R^{b} are independently selected from H; R⁹; OR⁹; NR¹⁰R¹¹; CN; C(O)R¹²; C(O)OR¹³; OC(O)R¹⁴; C(O)NR¹⁰R¹¹; and NHC(O)R¹⁵; wherein R⁹, R¹⁰, R¹¹, R¹², R¹³, R¹⁴, and R¹⁵ are independently selected from H and alkyl, alkenyl, and cycloalkyl groups containing 1-6 carbon atoms and optionally containing one or halogen atoms;
n is selected from an integer in the range of 0-100;
p is an integer of 0-10; and
q is an integer of 0-50.

2. The compound of claim 1, wherein R^{2a} is selected from amine groups, thiol groups, carboxylic acid-containing groups, ester-containing groups, aldehyde-containing groups, amine-reactive groups, thiol-reactive groups, carboxylic acid-reactive groups, ester-reactive groups, aldehyde-reactive groups, click reaction reactive groups, and affinity binding groups.

3. The compound of claim 1, wherein R^{2a} is a click reaction reactive group.

4. The compound of any one of claims 2-3, wherein the click reaction reactive group is selected from the group consisting of alkyne-containing group, azide, thiol, maleimide, vinylsulfone, pyridyl disulfide, alkene-containing group, and tetrazine-containing group.

5. The compound of any one of claims 2-3, wherein the click reaction reactive group is selected from alkyne-containing and azide groups, preferably an alkyne-containing group.

6. The compound of any one of claims 1-5, wherein n is 0.

7. The compound of any one of claims 1-5, wherein n is 1-100.

8. The compound of any one of claims 1-7, wherein p is 0, 1, or 2.

9. The compound of any one of claims 1-7, wherein p is 1.

10. The compound of any one of claims 1-9, wherein L³ comprises S—S.

11. The compound of any one of claims 1-7 and 10, wherein p is 1 or 2 and L⁴ is OC(O)O—Ar.

12. The compound of any one of claims 1-11, wherein L⁵ is selected from C(O)NH, C(O)O, OC(O)NH, OC(O)O, or NHC(O)NH, or L⁵ is a linker in which CH₂ is bound to any of the foregoing linkers.

13. The compound of any one of claims 1-12, wherein L⁵ is selected from C(O)O and OC(O)O, or L⁵ is a linker in which CH₂ is bound to any of the foregoing linkers.

14. The compound of any one of claims 1-13, wherein said aromatic or aliphatic ring or polycyclic ring system in R¹ comprises a phenyl ring.

15. The compound of any one of claims 1-14, wherein L² is C(O)O or C(O)NH.

16. The compound of any one of claims 1, 6-9, 11, and 14, wherein R^{2a} is an alkyne-containing group; L¹ is C(O); L² is C(O)O or C(O)NH; L³ is S—S; and L⁵ is selected from C(O)O and OC(O)O, or L⁵ is a linker in which CH₂ is bound to any of the foregoing linkers.

17. A compound having the following structure: 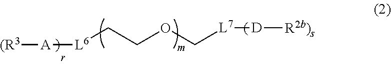
wherein:
R^{2b} is a second reactive group that forms a linkage with a first reactive group;
r and s are independently selected from an integer in the range of 1-6;
L⁶ is either: (i) a non-branching linker selected from CH₂, C(O), NH, O, S, C(O)NH, C(O)O, OC(O)NH, OC(O)O, and NHC(O)NH, or one or more of these attached together, wherein r is 1; or (ii) a branching linker selected from C, CH, N, B, or a linear or branched, cyclic or acyclic hydrocarbon linker group R^{o} containing 2-50 carbon atoms on which an r number of R³-A arms are attached, wherein Re optionally contains one or more heteroatoms selected from N, O, and S; or (iii) one or more non-branching linkers in (i) attached to a branching linker (ii);
L⁷ is either: (i) a non-branching linker selected from CH₂, C(O), NH, O, S, C(O)NH, C(O)O, OC(O)NH, OC(O)O, and NHC(O)NH, in which case s is 1; or (ii) a branching linker selected from C, CH, N, B, or a linear or branched, cyclic or acyclic hydrocarbon linker group Re containing 2-50 carbon atoms on which an s number of R^{2b}-D arms are attached, wherein Re optionally contains one or more heteroatoms selected from N, O, and S, or (iii) one or more non-branching linkers in (i) attached to a branching linker (ii);
A is a bond or a linker containing 1-50 carbon atoms and optionally containing one or more heteroatoms selected from N, O, and S;
D is a bond or a linker containing 1-50 carbon atoms and optionally containing one or more heteroatoms selected from N, O, and S;
R³ is independently a linear or branched, cyclic or acyclic, saturated or unsaturated hydrocarbon group containing 4-30 carbon atoms optionally containing one or more heteroatoms selected from N, O, S, and B and which functions as a cell penetration moiety;
and
m is selected from an integer in the range of 0-100.

18. The compound of claim 17, wherein R^{2b} is selected from amine groups, thiol groups, carboxylic acid-containing groups, ester-containing groups, aldehyde-containing groups, amine-reactive groups, thiol-reactive groups, carboxylic acid-reactive groups, ester-reactive groups, aldehyde-reactive groups, click reaction reactive groups, and affinity binding groups.

19. The compound of claim 17, wherein R^{2b} is a click reaction reactive group.

20. The compound of any one of claims 18 and 19, wherein the click reaction reactive group is selected from the group consisting of alkyne-containing group, azide, thiol, maleimide, vinylsulfone, pyridyl disulfide, alkene-containing group, and tetrazine-containing group.

21. The compound of any one of claims 18 and 19, wherein the click reaction reactive group is selected from alkyne-containing and azide groups, preferably an azide.

22. The compound of any one of claims 17-21, wherein m is 1-10.

23. The compound of any one of claims 17-22, wherein r is 2-6 and s is 1.

24. The compound of any one of claims 17-22, wherein r is 2 or 3 and s is 1.

25. The compound of any one of claims 17-22, wherein r is 1 and s is 2-6.

26. The compound of any one of claims 17-22, wherein r is 1 and s is 2 or 3.

27. The compound of any one of claims 17-26, wherein R³ is a saturated or unsaturated fatty acyl group.

28. The compound of any one of claims 17-26, wherein R³ is cholesterol or a derivative thereof.

29. The compound of any one of claims 17-28, wherein at least one of A and D contains a polyalkylene oxide segment.

30. The compound of any one of claims 17 and 29, wherein R^{2b} is an azide; r is 1, 2 or 3; s is 1; R³ is a saturated or unsaturated fatty acyl group; and m is 1-10.

31. The compound of any one of claims 17 and 29, wherein R^{2b} is an azide; r is 1, 2 or 3; s is 1; R³ is cholesterol or a derivative thereof; and m is 1-10.

32. The compound of any one of claims 17 and 29, wherein R^{2b} is an azide; r is 1; sis 1, 2 or 3; R³ is a saturated or unsaturated fatty acyl group; and m is 1-10.

33. The compound of any one of claims 17 and 29, wherein R^{2b} is an azide; r is 1; sis 1, 2 or 3; R³ is cholesterol or a derivative thereof; and m is 1-10.

34. A compound having the following structure: 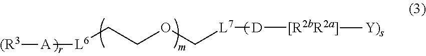
wherein Y= 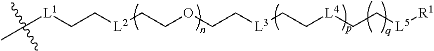
wherein:
R¹, R³, A, D, L¹, L², L³, L⁴, L⁵, L⁶, L⁷, r, m, s, n, p, and q are as defined in claims 1 and 16, and [R^{2b}R^{2a}] represents a reaction product between R^{2b} and R^{2a}.

35. The compound of claim 34, wherein [R^{2b}R^{2a}] comprises a reaction product between click reaction groups; amine and amine-reactive groups; thiol and thiol-reactive groups; carboxylic acid-containing groups and carboxylic acid-reactive groups; ester-containing groups and ester-reactive groups; or aldehyde-containing groups and aldehyde-reactive groups.

36. The compound of claim 34, wherein [R^{2b}R^{2a}] comprises a click reaction product, such as a 1,2,3-triazole.

37. The compound of any one of claims 34-36, wherein r is 1 or 2-6 and s is 1.

38. The compound of any one of claims 34-36, wherein r is 1 and sis 1 or 2-6.

39. The compound of any one of claims 34-38, wherein R³ is a saturated or unsaturated fatty acyl group.

40. The compound of any one of claims 34-38, wherein R³ is cholesterol or a derivative thereof.

41. The compound of any one of claims 34-40, wherein at least one of A and D contains a polyalkylene oxide segment.

42. The compound of any one of claims 34-41, wherein m is 1-10.

43. The compound of any one of claims 34 and 37-42, wherein [R^{2b}R^{2a}] comprises a 1,2,3-triazole; L¹ is C(O); L² is C(O)O or C(O)NH; L³ is S—S; and L⁵ is selected from C(O)O and OC(O)O, or L⁵ is a linker in which CH₂ is bound to any of the foregoing linkers.

44. The compound of any one of claims 34 and 41, wherein [R^{2b}R^{2a}] comprises a 1,2,3-triazole; r is 2 or 3; s is 1; R³ is a saturated or unsaturated fatty acyl group; and m is 1-10.

45. The compound of any one of claims 34 and 41, wherein [R^{2b}R^{2a}] comprises a 1,2,3-triazole; r is 2 or 3; sis 1; R³ is cholesterol or a derivative thereof; and m is 1-10.

46. The compound of any one of claims 34 and 41, wherein [R^{2b}R^{2a}] comprises a 1,2,3-triazole; r is 1; s is 2 or 3; R³ is a saturated or unsaturated fatty acyl group; and m is 1-10.

47. The compound of any one of claims 34 and 41, wherein [R^{2b}R^{2a}] comprises a 1,2,3-triazole; r is 1; s is 2 or 3; R³ is cholesterol or a derivative thereof; and m is 1-10.

48. A conjugate composition having the following structure: 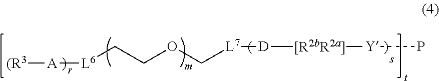
wherein Y′ is a linkage having the following structure: 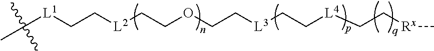
wherein:
R¹, R³, A, D, L¹, L², L³, L⁴, L⁵, L⁶, L⁷, r, m, s, n, p, and q are as defined in claims 1 and 16, and [R^{2b}R^{2a}] represents a reaction product between R^{2b} and R^{2a}; P is a peptide-containing or nucleic acid-containing molecule;
R^{x} is a linkage reaction product between L⁵-R¹, as defined in claim 1, and a group or domain on the P molecule;
the dashed bond indicates a covalent or non-covalent bond; and
t is at least 1.

49. The conjugate composition of claim 48, wherein P is a peptide or protein.

50. The conjugate composition of claim 49, wherein the peptide or protein contains at least 50 amino acids.

51. The conjugate composition of claim 49, wherein the peptide or protein contains at least 75 amino acids.

52. The conjugate composition of claim 49, wherein the peptide or protein contains at least 100 amino acids.

53. The conjugate composition of claim 49, wherein the peptide or protein contains less than 50 amino acids.

54. The conjugate composition of claim 49, wherein the peptide or protein has a molecular weight of 10-500 kDa.

55. The conjugate composition of claim 49, wherein the peptide protein has a molecular weight of 30-300 kDa, 50-300 kDa, 30-250 kDa, or 50-250 kDa.

56. The conjugate composition of any one of claims 48-55, wherein [R^{2b}R^{2a}] comprises a click reaction product, such as a 1,2,3-triazole.

57. The conjugate composition of any one of claims 48-55, wherein r is 1 or 2-6 and s is 1.

58. The conjugate composition of any one of claims 48-55, wherein r is 1 and s is 1 or 2-6.

59. The conjugate composition of any one of claims 48-58, wherein R³ is a saturated or unsaturated fatty acyl group.

60. The conjugate composition of any one of claims 48-58, wherein R³ is cholesterol or a derivative thereof.

61. The conjugate composition of any one of claims 48-60, wherein at least one of A and D contains a polyalkylene oxide segment.

62. The conjugate composition of any one of claims 48-61, wherein m is 1-10.

63. The conjugate composition of any one of claims 48-55 and 57-62, wherein [R^{2b}R^{2a}] comprises a click reaction product; L¹ is C(O); L² is C(O)O or C(O)NH; L³ is S—S; and L⁵ is selected from C(O)O and OC(O)O, or L⁵ is a linker in which CH₂ is bound to any of the foregoing linkers.

64. The conjugate composition of any one of claims 48-55 and 61, wherein [R^{2b}R^{2a}] comprises a click reaction product; r is 2 or 3; s is 1; R³ is a saturated or unsaturated fatty acyl group; and m is 1-10.

65. The conjugate composition of any one of claims 48-55 and 61, wherein [R^{2b}R^{2a}] comprises a click reaction product; r is 2 or 3; sis 1; R³ is cholesterol or a derivative thereof; and m is 1-10.

66. The conjugate composition of any one of claims 48-55 and 61, wherein [R^{2b}R^{2a}] comprises a click reaction product; r is 1; s is 2 or 3; R³ is a saturated or unsaturated fatty acyl group; and m is 1-10.

67. The conjugate composition of any one of claims 48-55 and 61, wherein [R^{2b}R^{2a}] comprises a click reaction product; r is 1; s is 2 or 3; R³ is cholesterol or a derivative thereof; and m is 1-10.

68. A conjugate composition having the following structure: 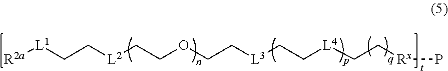
wherein:
R^{2a}, L¹, L², L³, L⁴, n, p, and q are as defined in claim 1;
P is a peptide-containing or nucleic acid-containing molecule;
R^{x} is a linkage reaction product between L⁵-R¹, as defined in claim 1, and a group on the P molecule;
the dashed bond indicates a covalent or non-covalent bond; and
t is at least 1.

69. The conjugate composition of claim 68, wherein P is a peptide or protein.

70. The conjugate composition of claim 69, wherein the peptide or protein contains at least 50 amino acids.

71. The conjugate composition of claim 69, wherein the peptide or protein contains at least 75 amino acids.

72. The conjugate composition of claim 69, wherein the peptide or protein contains at least 100 amino acids.

73. The conjugate composition of claim 69, wherein the peptide or protein contains less than 50 amino acids.

74. The conjugate composition of claim 69, wherein the peptide or protein has a molecular weight of 10-500 kDa.

75. The conjugate composition of claim 69, wherein the peptide or protein has a molecular weight of 30-300 kDa, 50-300 kDa, 30-250 kDa, or 50-250 kDa.

76. A method of producing the molecule of Formula (3) in claim 32, the method comprising:
reacting a molecule of the following formula: 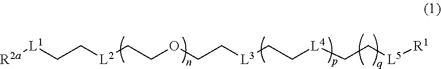
with a molecule of the following formula: 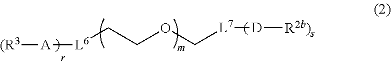
under conditions in which R^{2a} forms a linkage with R^{2b} to result in the following molecule: 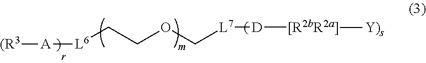
wherein Y= 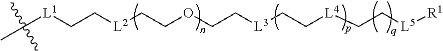
wherein R¹, R³, A, D, L¹, L², L³, L⁴, L⁵, L⁶, L⁷, r, m, s, n, p, and q are as defined in claims 1 and 16, and [R^{2b}R^{2a}] represents a reaction product between R^{2b} and R^{2a}.

77. The compound of claim 76, wherein [R^{2b}R^{2a}] comprises a reaction product between click reaction groups; amine and amine-reactive groups; thiol and thiol-reactive groups; carboxylic acid-containing groups and carboxylic acid-reactive groups; ester-containing groups and ester-reactive groups; or aldehyde-containing groups and aldehyde-reactive groups.

78. The method of claim 76, wherein [R^{2b}R^{2a}] comprises a click reaction product, such as a 1,2,3-triazole.

79. A method of producing a conjugate composition of the Formula (4) in claim 48 by a one-step or two-step approach, the method comprising:
for the one-step approach:
reacting a payload molecule (P) with a molecule of the following formula: 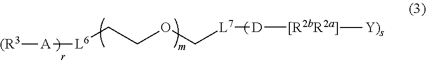
wherein Y= 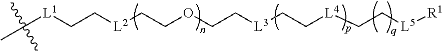
under conditions in which R¹ reacts with a group in P to result in the following molecule: 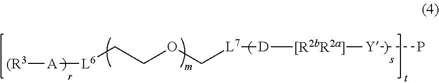
wherein Y′ is a linkage having the following structure: 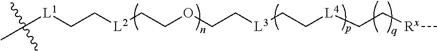
wherein:
R¹, R³, A, D, L¹, L², L³, L⁴, L⁵, L⁶, L⁷, r, m, s, n, p, and q are as defined in claims 1 and 16, and [R^{2b}R^{2a}] represents a reaction product between R^{2b} and R^{2a},
P is a peptide-containing or nucleic acid-containing molecule;
R^{x} is a linkage reaction product between L⁵-R¹, as defined in claim 1, and a group on the P molecule;
the dashed bond indicates a covalent or non-covalent bond; and
t is at least 1;
or for the two-step approach:
in a first step, reacting a payload molecule (P) with a molecule of the following formula: 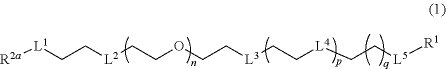
under conditions in which R¹ reacts with a group or domain in P to result in the following molecule: 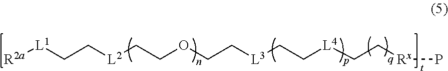
wherein:
R^{2a}, L¹, L², L³, L⁴, n, p, and q are as defined in claim 1;
P is a peptide-containing or nucleic acid-containing molecule;
R^{x} is a linkage reaction product between L⁵-R¹, as defined in claim 1, and a group on the P molecule;
the dashed bond indicates a covalent or non-covalent bond; and
t is at least 1; and
in a second step, reacting the conjugate molecule of Formula (5) with a molecule of the Formula (2) having the following structure: 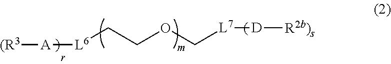
under conditions in which R^{2a} (a second reactive group) forms a linkage with R^{2b} to result in the following molecule of Formula (4): 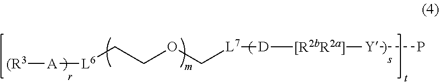
wherein Y′ is a linkage having the following structure: 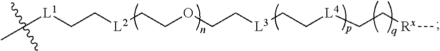
wherein:
R¹, R³, A, D, L¹, L², L³, L⁴, L⁵, L⁶, L⁷, r, m, s, n, p, and q are as defined in claims 1 and 16, and [R^{2b}R^{2a}] represents a reaction product between R^{2b} and R^{2a}; P is a peptide-containing or nucleic acid-containing molecule;
R^{x} is a linkage reaction product between L⁵-R¹, as defined in claim 1, and a group on the P molecule;
the dashed bond indicates a covalent or non-covalent bond; and
t is at least 1.

80. The compound of claim 79, wherein [R^{2b}R^{2a}] comprises a reaction product between click reaction groups; amine and amine-reactive groups; thiol and thiol-reactive groups; carboxylic acid-containing groups and carboxylic acid-reactive groups; ester-containing groups and ester groups; or aldehyde-containing groups and aldehyde-reactive groups.

81. The method of claim 79, wherein [R^{2b}R^{2a}] comprises a click reaction product, such as a 1,2,3-triazole.

82. The method of any one of claims 79-81, wherein P is a peptide or protein.

83. The method of claim 82, wherein the peptide or protein contains at least 50 amino acids.

84. The method of claim 82, wherein the peptide or protein contains at least 75 amino acids.

85. The method of claim 82, wherein the peptide or protein contains at least 100 amino acids.

86. The method of claim 82, wherein the peptide or protein contains less than 50 amino acids.

87. The method of claim 82, wherein the peptide or protein has a molecular weight of 10-500 kDa.

88. The method of claim 82, wherein the peptide or protein has a molecular weight of 30-300 kDa, 50-300 kDa, 30-250 kDa, or 50-250 kDa.

89. A method of producing a conjugate composition of the Formula (5) in claim 68, the method comprising:
reacting a payload molecule (P) with a molecule of the following formula: 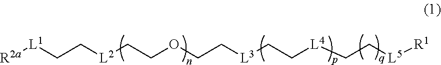
under conditions in which R¹ reacts with a group in P to result in the following molecule: 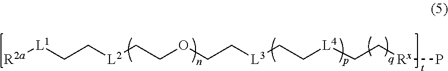
wherein:
R^{2a}, L¹, L², L³, L⁴, n, p, and q are as defined in claim 1;
P is a peptide-containing or nucleic acid-containing molecule;
R^{x} is a linkage reaction product between L⁵-R¹, as defined in claim 1, and a group on the P molecule;
the dashed bond indicates a covalent or non-covalent bond; and
t is at least 1.

90. The method of claim 89, wherein P is a peptide or protein.

91. The method of claim 90, wherein the peptide or protein contains at least 50 amino acids.

92. The method of claim 90, wherein the peptide or protein contains at least 75 amino acids.

93. The method of claim 90, wherein the peptide or protein contains at least 100 amino acids.

94. The method of claim 90, wherein the peptide or protein contains less than 50 amino acids.

95. The method of claim 90, wherein the peptide or protein has a molecular weight of 10-500 kDa.

96. The method of claim 90, wherein the peptide or protein has a molecular weight of 30-300 kDa, 50-300 kDa, 30-250 kDa, or 50-250 kDa.

97. A method of delivering a payload (P) to a cell, the method comprising contacting or interacting a conjugate molecule of the Formula (4) with a cell with resulting penetration of the conjugate molecule of the Formula (4) into the cell by means of the cell penetration moiety R³, wherein the conjugate molecule of Formula (4) has the following formula: 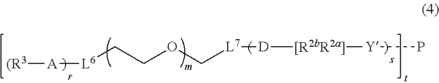
wherein Y′ is a linkage having the following structure: 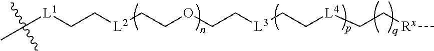
wherein:
R¹, R³, A, D, L¹, L², L³, L⁴, L⁵, L⁶, L⁷, r, m, s, n, p, and q are as defined in claims 1 and 16, and [R^{2b}R^{2a}] represents a reaction product between R^{2b} and R^{2a}; P is a peptide-containing or nucleic acid-containing molecule;
R^{x} is a linkage reaction product between L⁵-R¹, as defined in claim 1, and a group on the P molecule;
the dashed bond indicates a covalent or non-covalent bond; and
t is at least 1.

98. The method of claim 97, wherein [R^{2b}R^{2a}] comprises a reaction product between click reaction groups; amine and amine-reactive groups; thiol and thiol-reactive groups; carboxylic acid-containing groups and carboxylic acid-reactive groups; ester-containing groups and ester groups; or aldehyde-containing groups and aldehyde-reactive groups.

99. The method of claim 97, wherein [R^{2b}R^{2a}] comprises a click reaction product, such as a 1,2,3-triazole.

100. The method of any one of claims 97-99, wherein P is a peptide or protein.

101. The method of claim 100, wherein the peptide or protein contains at least 50 amino acids.

102. The method of claim 100, wherein the peptide or protein contains at least 75 amino acids.

103. The method of claim 100, wherein the peptide or protein contains at least 100 amino acids.

104. The method of claim 100, wherein the peptide or protein contains less than 50 amino acids.

105. The method of claim 100, wherein the peptide or protein has a molecular weight of 10-500 kDa.

106. The method of claim 100, wherein the peptide or protein has a molecular weight of 30-300 kDa, 50-300 kDa, 30-250 kDa, or 50-250 kDa.

107. A pharmaceutical composition comprising a conjugate of any one of claims 48-75 and a pharmaceutically acceptable carrier.

108. A method of delivering a peptide-containing or nucleic acid-containing payload molecule (P) to a subject in need thereof, the method comprising administering the conjugate of any one of claims 48-75 or the pharmaceutical composition of claim 107 to the subject.

109. The method of claim 108, wherein P is a peptide or protein.

110. The method of claim 108, wherein P is a nucleic acid-containing molecule, such as a DNA, RNA, or oligonucleotide.

111. A method of treating a disease or condition in a subject in need thereof, the method comprising administering a conjugate of any one of claims 48-75 or the pharmaceutical composition of claim 107 to the subject wherein the payload (P) in the conjugate is a peptide-containing or nucleic acid-containing molecule.

112. The method of claim 111, wherein P is a nucleic acid-containing molecule.

113. The method of claim 111, wherein the nucleic acid-containing molecule functions as gene editing machinery.

114. The method of claim 111, wherein P is a peptide or protein.

115. The method of claim 114, wherein the peptide or protein contains at least 50 amino acids.

116. The method of claim 114, wherein the peptide or protein contains at least 75 amino acids.

117. The method of claim 114, wherein the peptide or protein contains at least 100 amino acids.

118. The method of claim 114, wherein the peptide or protein contains less than 50 amino acids.

119. The method of claim 114, wherein the peptide or protein has a molecular weight of 10-500 kDa.

120. The method of claim 114, wherein the peptide or protein has a molecular weight of 30-300 kDa, 50-300 kDa, 30-250 kDa, or 50-250 kDa.

121. The method of any one of claims 111-120, comprising administering the conjugate or pharmaceutical formulation to one or more cells, tissues, or organs of the subject.

122. The method of any one of claims 111-120, wherein administration is in vitro or in vivo.

123. The method of any one of claims 111-120, wherein the subject exhibits one or more signs or symptoms associated with a genetic neuropathy, a genetic based musculopathy, a genetic eye disease or disorder, a genetic lung disease or disorder, a genetic liver disease or disorder, neurodegenerative disease, or cancer.

124. The method of any one of claims 111-123, wherein the subject exhibits one or more signs or symptoms associated with Angelman syndrome, HIST1H1E (H₁-4) syndrome, Prader-Willi syndrome, Alzheimer's disease, Huntington's disease, Parkinson's Disease (PD), Multiple Sclerosis (MS), Cerebral Palsy (CP), Spinocerebellar Ataxias, Pick's disease, Gerstmann-Straussler-Scheinker disease, Hallervorden-Spatz disease, Jakob-Creutzfeldt disease, dystonia, amyotrophic lateral sclerosis, muscular atrophies, muscular dystrophies (such as Duchenne, Becker, facioscapulohumeral, myotonic, congenital, distal, Emery-Dreifuss, oculopharyngeal, and limb girdle), congenital myopathies (such as central core, Myotubular, Nemaline, Ullrich/Bethlem, and RyR1), metabolic disorders (such as Pompe's disease), Retinitis pigmentosa, choroideremia, Stargardt disease, cone-rod dystrophy, Leber congenital amaurosis, cystic fibrosis, primary ciliary dyskinesia, alpha-1-antitrypsin deficiency, surfactant metabolism dysfunction 1-4, familial pulmonary fibrosis, pulmonary alveolar microlithiasis, Dyskeratosis congenita, neurofibromatosis type I, tuberous sclerosis/LAM, Birt-Hogg-Dubé Syndrome, Hyper IgE syndrome, Hermansky-Pudlak syndrome, Gaucher disease type I, Niemann-Pick disease type B, and Lysinuric protein intolerance, hemochromatosis, Wilsons disease, alpha-1-antitrypsin deficiency, or cancer.

125. The method of any one of claims 111-123, wherein the subject exhibits one or more signs or symptoms associated with Angelman syndrome, HIST1H1E (H₁-4) syndrome, Prader-Willi syndrome, Alzheimer's disease, Huntington's disease, Parkinson's Disease (PD), Multiple Sclerosis (MS), Cerebral Palsy (CP), Spinocerebellar Ataxias, Pick's disease, Gerstmann-Straussler-Scheinker disease, Hallervorden-Spatz disease, or Jakob-Creutzfeldt disease.

126. The method of any one of claims 111-123, wherein the subject exhibits one or more signs or symptoms associated with Angelman syndrome, HIST1H1E (H₁-4) syndrome, or Prader-Willi syndrome.

127. The method of any one of claims 111-122, wherein the subject has a neurodegenerative disease.

128. The method of any one of claims 111-112, wherein the subject has cancer.
